(19) United States
(12) Patent Application Publication
(10) Pub. No.: US 2022/0339179 A1
(43) Pub. Date: Oct. 27, 2022

(54) TREATMENT OF HR DEFICIENT CANCER

(71) Applicant: THE FRANCIS CRICK INSTITUTE LIMITED, London (GB)

(72) Inventors: Kasper Fugger, London (GB); Stephen West, London (GB)

(21) Appl. No.: 17/640,706

(22) PCT Filed: Sep. 9, 2020
(86) PCT No.: PCT/EP2020/075245

§ 371 Date: Mar. 4, 2022

(30) Foreign Application Priority Data
Sep. 10, 2019 (GB) 1913030.1

(51) Int. Cl. A61K 31/7072; A61K 31/502; A61K 31/55; A61K 31/454; A61K 31/5025; A61K 31/4184; A61K 31/5517; A61K 31/496; C12N 15/113; A61P 35/00

(52) U.S. Cl. CPC A61K 31/7072; A61K 31/502; A61K 31/55; A61K 31/454; A61K 31/5025; A61K 31/4184; A61K 31/5517; A61K 31/496; C12N 15/1137; A61P 35/00; C12N 2310/14; C12N 2310/531; C12N 2310/11

(57) ABSTRACT

This invention relates to the finding that homologous recombination (HR) deficient cells are sensitised to PARP inhibition by either (i) catalytic inhibition or genetic ablation of 2′-deoxynucleoside 5′-phosphate N-hydrolase 1 (DNPH1) or (ii) administration of a substrate of DNPH1, such as 5-hydroxymethyl-deoxyuridine (hmdU). The invention also relates to the finding that catalytic inhibition or genetic ablation of DNPH1 combined with administration of hmdU causes synthetic lethality in HR deficient cells in the absence of PARP inhibition. Methods and compounds for use in the treatment of HR deficient cancer are provided.

### FIELD

The present invention relates to the treatment of cancers that are deficient in homologous recombination (HR), and in particular to the sensitisation of HR deficient cancers to inhibition of poly (ADP-ribose) polymerase (PARP).

### BACKGROUND

The genome is constantly exposed to DNA damage by both exogenous sources as well as endogenously by reactive oxygen species and replication-inflicted errors that are causes of genome instability and cancer. Homologous recombination repair (HRR) constitutes one of the main pathways which promotes error-free repair of various DNA lesions. The breast cancer genes, BRCA1 and BRCA2 (hereafter BRCA), are key components of the HRR pathway and inherited mutations in either of these genes predispose to the development of breast and ovarian cancer. Cancer cells with BRCA deficiency are hypersensitive to treatment with PARP inhibitors which is exploited in treatment of HRR deficient tumours (Bryant et al (2005) Nature 434 913-917; Farmer et al (2005) Nature 434 917-921). The synthetic lethality is thought to be caused by trapping of PARP1 on various types of lesions, including DNA single-strand breaks (Zimmerman et al (2018) Nature 559 285-289). PARP inhibitors olaparib and rucaparib have been approved for the treatment of BRCA deficient ovarian cancer, niraparib has been approved for the treatment of epithelial ovarian, fallopian tube and primary peritoneal cancer and talazoparib has been approved for the treatment of BRCA deficient breast cancer. Further clinical trials are ongoing.

Treatment with PARP inhibitors is associated with moderate toxicity in patients. Furthermore, although the initial response rate is very high, cancers treated with PARP inhibitors eventually develop resistance to the treatment, for example by restoring HR activity through the inactivation of one or more factors in the 53BP1 pathway, or by the inactivation of PARP1 or PARG (Noordermeer et al (2019) Trends in Cell Biology). Reducing the toxicity and/or tumour resistance would facilitate the use of PARP inhibition to treat cancer.

### SUMMARY

The present inventors have discovered that either catalytic inhibition or genetic ablation of 2′-deoxynucleoside 5′-phosphate N-hydrolase 1 (DNPH1) or administration of a substrate of DNPH1, such as 5-hydroxymethyl-deoxyuridine (hmdU), sensitizes HR deficient cells to PARP inhibition. Catalytic inhibition or genetic ablation of DNPH1 combined with administration of hmdU were also found to cause synthetic lethality in HR deficient cells in the absence of PARP inhibition. These findings may be useful for example in the treatment of HR deficient cancer.

A first aspect of the invention provides a method of treating an HR deficient cancer comprising reducing 2′-deoxynucleoside 5′-phosphate N-hydrolase 1 (DNPH1) activity in an individual in need thereof and administering a poly (ADP-ribose) polymerase (PARP) inhibitor to the individual.

Reduction of DNPH1 activity and administration of the PARP inhibitor may be performed in any order or simultaneously.

A second aspect of the invention provides a method of screening for a compound useful in sensitising an HR deficient cancer in a patient to treatment with a PARP inhibitor, the method comprising; determining the expression or activity of DNPH1 in the presence or absence of a test compound.

A decrease in DNPH1 expression or activity in the presence relative to the absence of the test compound may be indicative that the test compound is useful in sensitising an HR deficient cancer in a patient to treatment with a PARP inhibitor.

A third aspect of the invention provides a method of sensitising a HR deficient cancer in an individual to treatment with a PARP inhibitor comprising reducing DNPH1 activity in the individual. Reducing DNPH1 activity in the individual sensitises the HR deficient cancer to treatment with the PARP inhibitor.

A fourth aspect of the invention provides a method of sensitising a HR deficient cancer in an individual to a reduction in 2′-deoxynucleoside 5′-phosphate N-hydrolase 1 (DNPH1) activity comprising administering a PARP inhibitor to the individual. Administration of the PARP inhibitor sensitises the HR deficient cancer to the reduction in DNPH1 activity.

A fifth aspect of the invention provides a method of treating an HR deficient cancer comprising; administering a combination of PARP inhibitor and a 5-modified-2′-deoxypyrimidine nucleoside to an individual in need thereof.

Suitable 5-modified-2′-deoxpyrimidine nucleosides include 5-hydroxymethyl-2′-deoxyuridine (hmdU), 5-formyl-2′-deoxyuridine (fodU) and 5-hydroxymethyl-2′-deoxycytidine (hmdC),

A sixth aspect of the invention provides a method of sensitising an HR deficient cancer in an individual to treatment with a PARP inhibitor comprising administering a 5-modified-2′-deoxypyrimidine nucleoside to the individual. Administration of the 5-modified-2′-deoxypyrimidine nucleoside to the individual sensitises the HR deficient cancer to treatment with the PARP inhibitor

A seventh aspect of the invention provides a method of sensitising an HR deficient cancer in an individual to treatment with a 5-modified-2′-deoxypyrimidine nucleoside comprising administering a PARP inhibitor to the individual. Administration of the PARP inhibitor to the individual sensitises the HR deficient cancer to treatment with the 5-modified-2′-deoxpyrimidine nucleoside.

An eighth aspect of the invention provides a method of treating an HR deficient cancer comprising administering a 5-modified-2′-deoxypyrimidine nucleoside to an individual in need thereof and reducing 2′-deoxynucleoside 5′-phosphate N-hydrolase 1 (DNPH1) activity in the individual.

Reduction of DNPH1 activity and administration of the 5-modified-2′-deoxypyrimidine nucleoside may be performed in any order or simultaneously.

A ninth aspect of the invention provides a method of sensitising a HR deficient cancer in an individual to treatment with a 5-modified-2′-deoxypyrimidine nucleoside comprising reducing DNPH1 activity in the individual.

Reducing DNPH1 activity sensitises the HR deficient cancer to treatment with the 5-modified-2′-deoxypyrimidine nucleoside.

A tenth aspect of the invention provides a method of sensitising a HR deficient cancer in an individual to a reduction in 2′-deoxynucleoside 5′-phosphate N-hydrolase 1 (DNPH1) activity comprising; administering a 5-modified-2′-deoxypyrimidine nucleoside to the individual. Administration of a 5-modified-2′-deoxypyrimidine nucleoside sensitises the HR deficient cancer to a reduction in 2′-deoxynucleoside 5′-phosphate N-hydrolase 1 (DNPH1) activity.

An eleventh aspect of the invention provides a method of screening for a compound useful in sensitising an HR deficient cancer in a patient to treatment with a 5-modified-2′-deoxypyrimidine nucleoside, the method comprising determining the activity of an DNPH1 protein in the presence or absence of a test compound.

A decrease in DNPH1 activity in the presence relative to the absence of the test compound is may be indicative that the test compound is useful in sensitising an HR deficient cancer in a patient to treatment with the 5-modified-2′-deoxypyrimidine nucleoside.

An HR deficient cancer of the eighth to the eleventh aspects may be resistant to PARP inhibition. For example, the HR deficient cancer may have developed PARP inhibition resistance following treatment with a PARP inhibitor.

A twelfth aspect of the invention provides a method of treating an HR deficient cancer comprising reducing 2′-deoxynucleoside 5′-phosphate N-hydrolase 1 (DNPH1) activity in an individual in need thereof and administering a poly (ADP-ribose) polymerase (PARP) inhibitor and a 5-modified-2′-deoxpyrimidine nucleoside to the individual.

Reduction of DNPH1 activity and administration of the PARP inhibitor and 5-modified-2′-deoxypyrimidine nucleoside may be performed in any order or simultaneously.

A thirteenth aspect of the invention provides a method of sensitising a HR deficient cancer in an individual to treatment with a PARP inhibitor comprising reducing DNPH1 activity in the individual and administering a 5-modified-2′-deoxypyrimidine nucleoside to the individual. Reducing DNPH1 activity in the individual and administering a 5-modified-2′-deoxypyrimidine nucleoside to the individual sensitises the HR deficient cancer to treatment with the PARP inhibitor.

A fourteenth aspect of the invention provides a method of ameliorating toxicity in an organ or tissue of an individual undergoing treatment with a PARP inhibitor comprising;
selectively reducing SMUG1 activity in the organ or tissue of the individual.

The individual may have an HR deficient cancer.

A fifteenth aspect of the invention provides a method of screening for a compound useful in ameliorating toxicity in individual undergoing treatment with a combination of a PARP inhibitor and a 5-modified-2′-deoxypyrimidine nucleoside comprising determining the expression or activity of SMUG1 in the presence or absence of a test compound, wherein a decrease in SMUG1 expression or activity in the presence relative to the absence of the test compound is indicative that the test compound is useful in ameliorating toxicity in individual undergoing treatment with a combination of a PARP inhibitor and a 5-modified-2′-deoxypyrimidine nucleoside.

The method may further comprise determining the effect of the test compound on the expression or activity of SMUG1 in tissue or organ relative to other tissue or organs in the individual.

A sixteenth aspect of the invention provides a PARP inhibitor for use in a method of treatment or sensitising according to the first, fourth, fifth, seventh or twelfth aspects.

A seventeenth aspect of the invention provides the use of a PARP inhibitor in the manufacture of a medicament for use in a method of treatment or sensitising according to the first, fourth, fifth, seventh or twelfth aspects.

An eighteenth aspect of the invention provides an agent that reduces DNPH1 activity for use in a method of treatment or sensitising according to the first, third, eighth, ninth, twelfth or thirteenth aspects.

A nineteenth aspect of the invention provides the use of an agent that reduces DNPH1 activity for the manufacture of a medicament for use in a method of treatment or sensitising according to the first, third, eighth, ninth, twelfth or thirteenth aspects.

A twentieth aspect of the invention provides a 5-modified-2′-deoxypyrimidine nucleoside for use in a method of treatment or sensitising according to the fifth, sixth, eighth, tenth, twelfth or thirteenth aspects.

A twenty first aspect of the invention provides the use of a 5-modified-2′-deoxpyrimidine nucleoside in the manufacture of a medicament for use in a method of treatment or sensitising according to the fifth, sixth, eighth, or tenth aspects.

A twenty second aspect of the invention provides an agent that selectively reduces SMUG1 activity in the bone marrow for use in a method of treatment or sensitising according to the twelfth aspect.

A twenty third aspect of the invention provides the use of an agent that selectively reduces SMUG1 activity in the manufacture of a medicament for use in a method of treatment or sensitising according to the according to the twelfth aspect.

Other aspects and embodiments of the invention are described in more detail below.

### BRIEF DESCRIPTION OF THE FIGURES

FIG. 1 is a Volcano plot showing sgRNA scores from MAGeCK analysis of genome-wide CRISPR-Cas9 olaparib dropout/enrichment screen. Each point represents limit fold change on the x-axis (sensitising sgRNAs to the left and resistance causing to the right) with corresponding MAGeCK score on the y-axis. Factors involved in BER and nucleotide metabolism are highlighted.

FIG. 2 shows that DNPH1 loss increases PARPi induced synthetic lethality in HR deficient MUS81−/− cells. eHAP WT or the indicated k/o cell lines were treated continuously for 6 days with various doses of olaparib. Cell viability was determined using CellTiter-Glo (mean with s.e.m.) (n=3). Data was analysed using ANOVA for multiple comparisons (MUS81−/− vs MUS81−/−DNPH1−/−, p=0.0013; MUS81−/− vs MUS81−/−ITPA−/−, p=0.0144).

FIG. 3 shows that DNPH1 loss increases synthetic lethality caused by PARP inhibition in HR deficient MUS81−/− cells. eHAP MUS81−/− cell lines were treated continuously for 6 days with various doses of olaparib (left), veliparib (middle) and talazoparib (right). Cell viability was determined using CellTiter-Glo (mean with s.e.m.) (n=3). Data was analysed using ANOVA for multiple comparisons.

FIG. 4 shows that DNPH1 loss increases synthetic lethality in BRCA2 deficient DLD1 cells. The indicated DLD1 cell lines were treated continuously for 10 days with various doses of olaparib (left) or veliparib (right). Cell viability was determined as above. Data was analysed using ANOVA for multiple comparisons (BRCA2−/− vs BRCA2−/−DNPH1−/−, p=0.0202).

FIG. 5 shows that DNPH1 loss increases PARPi induced synthetic lethality in BRCA1 deficient SUM149 cells. SUM149 WT or the indicated k/o cell lines were treated continuously for 6 days with various doses of olaparib. Cell viability was determined using CellTiter-Glo (mean with s.e.m.) (n=3). Data was analysed using ANOVA for multiple comparisons.

FIG. 6 also shows that DNPH1 loss increases PARPi or hmdU induced synthetic lethality in BRCA1 deficient SUM149 cells. The indicated SUM149 cell lines were treated continuously for 10 days with various doses of olaparib (left) or hmdU (right). Cell viability was determined as above. Data was analysed using ANOVA for multiple comparisons.

FIG. 7 shows the nucleoside composition of eHAP WT and DNPH1−/− cells. Genomic DNA was extracted from eHAP WT or DNPH1−/− cells, digested and analysed for its nucleoside composition by LC-MS. The ratio of the indicated nucleosides in DNPH1−/− vs WT genomic DNA (mean with s.e.m.) (n=7).

FIG. 8 shows a biochemical characterization of DNPH1 activity towards nucleoside monophosphate substrates. Wild-type DNPH1 or inactive DNPH1^{E105Q} (4 μM) were incubated individually with the indicated substrates (1 mM) for 45 min. Reaction products were analysed by RP-HPLC and visualized as chromatograms. Untreated nucleoside monophosphates and the nucleobase hmU are shown as standards.

FIG. 9 shows that DNPH1 loss sensitizes HR deficient cells to hmdU, fodU and hmdC. eHAP MUS81−/− and MUS81−/−DNPH1−/− cells were either left untreated or treated continuously with the indicated nucleosides (200 nM) for 6 days. Bar chart shows the ratio of cell viability between MUS81−/−DNPH1−/− vs MUS81−/− as determined using CellTiter-Glo (mean with s.e.m.) (n=3).

FIG. 10 shows that treatment with hmdU, fodU or hmdC nucleosides causes synthetic lethality with PARPi in HR deficient MUS81 KO eHAP cells.

FIG. 11 shows that treatment with hmdU causes synthetic lethality with PARPi in BRCA1 k/o SUM149 cells (11A) and BRCA2 k/o DLD1 cells (11B). Patient-derived SUM149 BRCA1mut (parental) and revertant (WT) cells were treated continuously for 8 days with olaparib (250 nM), hmdU (2 μM) or a combination (11A). DLD1 WT and BRCA2−/− cells were treated continuously for 10 days with olaparib (10 nM), hmdU (2 μM) or a combination (11B).

FIG. 12 shows that treatment with hmdU induces synthetic lethality with olaparib (left), veliparib (middle) and talazoparib (right) in HR deficient eHAP MUS81 k/o cells.

FIG. 13 shows that treatment with olaparib causes synthetic lethality with DNPH1 knockout in HR deficient MUS81 k/o eHAP cells and this synthetic lethality is rescued by DCTD knockout. eHAP cell lines were continuously treated with olaparib (50 nM) for 6 days.

FIG. 14 shows that loss of DCTD lowers genomic hmdU levels in both WT and DNPH1 k/o eHAP cells. Genomic DNA was extracted from eHAP WT, DNPH1−/− or DNPH1−/−DCTD−/− cells, digested and analysed for hmdU content by LC-MS. Bar chart shows the ratio of hmdU levels to WT (mean with s.e.m.) (n=3).

FIG. 15 shows that loss of DNPH1 is synthetic lethal with hmdU treatment in HR deficient BRCA1 k/o SUM149 cells (15A) and HR deficient BRCA2 k/o DLD1 cells (15B).

FIG. 16 shows that chemical inhibition of DNPH1i sensitises eHAP MUS81 k/o cells to hmdU. eHAP MUS81−/− cells were either untreated or treated with 250 nM hmdU in the presence or absence of DNPH1i for 6 days and cell viability was determined (mean with s.e.m.) (n=3).

FIG. 17 shows the effect of DNPH1 i on eHAP MUS81−/− and MUS81−/−DNPH1−/− k/o cells. Cells were either untreated or treated with hmdU in the presence or absence of DNPH1i for 6 days and cell viability was determined (mean with s.e.m.) (n=3).

FIG. 18 shows the killing of BRCA1 deficient cells by targeting DNPH1. SUM149 BRCA1mut (parental) and WT (revertant) cell lines were either left untreated or treated with olaparib in the absence (black lines) or presence of hmdU (2 μM; red lines) for 8 days. Cell viability was determined using CellTiter-Glo (mean with s.e.m.) (n=3). Blue dotted line represents EC50 values (FIG. 18A). SUM149 BRCA1mut (parental) and WT (revertant) cell lines were either left untreated or treated with the indicated doses of hmdU in the absence (black lines) or presence of DNPH1i (0.5 μM; red lines) for 8 days. (n=3) (FIG. 18B).

FIG. 19 shows a comparison of the therapeutic index of SUM149 BRCA1 mut cells. The ratio of EC50 values from (FIG. 18A) and (FIG. 18B) is shown.

FIG. 20 shows the killing of PARPi-resistant SUM149 BRCA1 mut/53BP1−/− or WT (revertant) cell lines by targeting DNPH1. SUM149 BRCA1mut/53BP1−/− or WT (revertant) cell lines were treated as in FIG. 18A. For direct comparison, SUM149 BRCA1 mut (parental) and WT (revertant) cell line curves from FIG. 18 are shown (solid red and black lines, respectively (see left panel). SUM149 BRCA1mut/53BP1−/− or WT (revertant) cell lines were treated as in FIG. 18B (right panel). For direct comparison, SUM149 BRCA1mut (parental) and WT (revertant) cell line curves from FIG. 18B are shown (solid red and black lines, respectively).

FIG. 21 shows a comparison of therapeutic index of SUM149 BRCA1mut/53BP1−/− cells. The ratio of EC50 values from the left and right panels of FIG. 20 is shown.

FIG. 22 shows the killing of PARPi-resistant SUM149 BRCA1mut/PARP1−/− or WT (revertant) cell lines by targeting DNPH1. Left panel shows SUM149 BRCA1mut/PARP1−/− or WT (revertant) cell lines were treated as in FIG. 18A. For direct comparison, SUM149 BRCA1mut (parental) and WI (revertant) cell line curves from FIG. 18A are shown (solid red and black lines, respectively). Right panel shows SUM149 BRCA1mut/PARP1−/− or WT (revertant) cell lines treated as in FIG. 18B. For direct comparison, SUM149 BRCA1mut (parental) and WT (revertant) cell line curves from FIG. 18B are shown (solid red and black lines, respectively).

FIG. 23 shows a comparison of therapeutic index of SUM149 BRCA1mut/PARP1−/− cells. The ratio of EC50 values from the left and right panels of FIG. 22 is shown.

FIG. 24 shows that loss of SMUG1 causes resistance to the combination of PARPi and hmdU in MUS81 k/o cells. eHAP WT, MUS81−/− and MUS81−/−SMUG1−/− cell lines were treated with olaparib (25 nM) and the indicated doses of hmdU for 6 days. Cell viability was determined as above (n=3). (MUS81−/− vs MUS81−/−SMUG1−/−, p=0.0014).

FIG. 25 shows that loss of SMUG1 causes resistance to the combination of PARPi and hmdU in BRCA2 deficient cells. DLD1 WT and BRCA2 k/o cell lines were treated with olaparib (10 nM) and hmdU (2 μM) for 10 days and cell viability determined (n=3).

FIG. 26 shows the results hmdU induced PARP trapping. eHAP WT and SMUG1−/− cells were either left untreated or pre-treated for 24 hours with hmdU (350 nM) or 2 hours with MMS (0.01%) following by addition of olaparib for 4 hours (10 μM). Samples were subjected to subcellular fractionation and the nuclear soluble or chromatin fractions were analysed by SDS-PAGE followed by immunoblotting with the indicated antibodies (n=3).

FIG. 27 shows a quantification DNA damage induced gammaH2AX foci. DLD1 cell lines were either untreated or treated with olaparib (10 nM), hmdU (100 nM) or in combination for 48 hours and subjected to immunofluorescence staining with a γH2AX antibody (red). DNA was stained with DAPI (blue). (n=4).

FIG. 28 shows a quantification of DNA damage induced RAD51 foci. DLD1 WT and k/o cell lines that were either left untreated or treated with olaparib (10 nM) and hmdU (100 nM) for 48 hours and subjected to immunofluorescence staining with a RAD51 antibody. DNA was stained by DAPI (blue) (n=4).

FIG. 29 shows model for hmdC metabolism and hmdU-induced cell death. To prevent the incorporation of salvaged nucleoside carrying epigenetic marks, such as hmdC, they are degraded in a two-step process. First, hmdCMP is deaminated to cytotoxic hmdUMP by DCTD, and second DNPH1 promotes the hydrolysis of hmdUMP into hmU and dRP. In the absence of DNPH1, hmdUMP levels increase and hmdUMP becomes phosphorylated by DTYMK and incorporated into DNA. SMUG1 glycosylase excises genomic hmdU, leading to PARP trapping, replication fork collapse and the death of HR-deficient cells following PARP inhibition. Combined DNPH1 inhibition and hmdU administration efficiently kills PARPi-resistant BRCA1 deficient cells.

### DETAILED DESCRIPTION

This invention relates to the finding that the potency of poly (ADP-ribose) polymerase (PARP) inhibition in homologous recombination (HR) deficient cells is dramatically increased by the inhibition or loss of 2′-Deoxynucleoside 5′-Phosphate N-Hydrolase 1 (DNPH1) or co-treatment with 5-hydroxymethyl-2′-deoxyuridine (hmdU). Furthermore, the inhibition DNPH1 in combination with hmdU treatment was found to be synthetically lethal in HR deficient cancer cells in the absence of PARP inhibition.

The term PARP as used herein refers to PARP1 (EC 2.4.2.30, Genbank No: M32721; Gene ID 142) unless context dictates otherwise. PARP1 is a chromatin-associated poly (ADP-ribose) polymerase that is involved in the cellular response to single strand DNA breaks. PARP1 may have the reference amino acid sequence of database accession number NP_001609.2 or a variant thereof and may be encoded by the nucleotide sequence of NM_001618.4 or a variant thereof. PARP1 is important for repairing single-strand breaks in DNA through the base excision repair pathway. If such nicks persist unrepaired until DNA is replicated (which must precede cell division), then the replication itself can cause double strand breaks to form.

An amino acid sequence as described herein may comprise the amino acid sequence of a reference human amino acid sequence, or an amino acid sequence having at least 80% sequence identity, at least 85% sequence identity, at least 90% sequence identity, at least 95% identity, or at least 98% identity to a reference human amino acid sequence. A nucleotide sequence as described herein may comprise a nucleotide sequence of a reference human coding sequence, or a nucleotide sequence having at least 80% sequence identity, at least 85% sequence identity, at least 90% sequence identity, at least 95% identity, or at least 98% identity to a reference human coding sequence.

Sequence identity is commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), SSEARCH (Smith and Waterman (1981) J. Mol Biol. 147: 195-197), HMMER3 (Johnson L S et al BMC Bioinformatics. 2010 Aug. 18; 110:431) or the TBLASTN program, of Altschul et al. (1990) supra, generally employing default parameters (see for example Pearson Curr Prot Bioinformatics (2013) 0 3 doi:10.1002/0471250953.bi0301s42). In particular, the psi-Blast algorithm may be used (Altschul et al. Nucl. Acids Res. (1997) 25 3389-3402). Sequence identity and similarity may also be determined using Genomequest™ software (Gene-IT, Worcester Mass. USA). Sequence comparisons are preferably made over the full-length of the relevant sequence described herein.

A PARP inhibitor is a compound or substance that inhibits the expression levels or biological activity of poly ADP ribose polymerase (PARP). A suitable PARP inhibitor may selectively inhibit PARP-1 with an IC50 of less than 20 nM, less than 10 nM, less than 5 nM or less than 2 nM in a cell free assay (Shen et al (2013) Clin Cancer Res 19 (18) 5003-5015).

Suitable assay for measuring the inhibition of PARP, including fluorescent and chemiluminescent assays, are well known in the art. For example, PARP inhibition may be measured by determining the inhibition of PARP mediated NAD+ depletion by coupling NAD+ levels to a cycling assay involving alcohol dehydrogenase and diaphorase which generates a fluorescent molecule, such as resorufin (see for example, Fluorescent Homogenous PARP inhibition Assay Kit Cat. #4690-096-K, Trevigen Inc MD USA).

PARP inhibition may cause multiple double strand breaks to form, and in cancer cells which are deficient in HR, these double strand breaks cannot be efficiently repaired, thereby leading to the death of the cells. As normal, non-cancerous cells do not replicate DNA as often as cancer cells, and generally have functional HR, normal cells survive PARP inhibition. In addition, a PARP inhibitor may trap PARP proteins at sites of DNA damage. The trapped PARP protein-DNA complexes are highly toxic to cells because they block DNA replication, further contributing to cell death.

Numerous examples of compounds which inhibit PARP are known and may be used as described herein, including:

1. Nicotinamides, such as 5-methyl nicotinamide and O-(2-hydroxy-3-piperidino-propyl)-3-carboxylic acid amidoxime, and analogues and derivatives thereof.

2. Benzamides, including 3-substituted benzamides such as 3-aminobenzamide, 3-hydroxybenzamide, 3-nitrosobenzamide, 3-methoxybenzamide and 3-chloroprocainamide, and 4-aminobenzamide, 1,5-di[(3-carbamoylphenyl)aminocarbonyloxy]pentane, and analogues and derivatives thereof.

3. Isoquinolinones and Dihydroisoquinolinones, including 2H-isoquinolin-1-ones, 3H-quinazolin-4-ones, 5-substituted dihydroisoquinolinones such as 5-hydroxy dihydroisoquinolinone, 5-methyl dihydroisoquinolinone, and 5-hydroxy isoquinolinone, 5-amino isoquinolin-1-one, 5-dihydroxyisoquinolinone, 3,4 dihydroisoquinolin-1(2H)-ones such as 3,4 dihydro-5-methoxy-isoquinolin-1(2H)-one and 3,4 dihydro-5-methyl-1(2H)isoquinolinone, isoquinolin-1(2H)-ones, 4,5-dihydro-imidazo[4,5,1-ij]quinolin-6-ones, 1,6-naphthyridine-5(6H)-ones, 1,8-naphthalimides such as 4-amino-1,8-naphthalimide, isoquinolinone, 3,4-dihydro-5-[4-1(1-piperidinyl) butoxy]-1(2H)-isoquinolinone, 2,3-dihydrobenzo[de]isoquinolin-1-one, 1-11b-dihydro-[2H]benzopyrano[4,3,2-de]isoquinolin-3-one, and tetracyclic lactams, including benzpyranoisoquinolinones such as benzopyrano[4,3,2-de]isoquinolinone, and analogues and derivatives thereof

4. Benzimidazoles and indoles, including benzoxazole-4-carboxamides, benzimidazole-4-carboxamides, such as 2-substituted benzoxazole 4-carboxamides and 2-substituted benzimidazole 4-carboxamides such as 2-aryl benzimidazole 4-carboxamides and 2-cycloalkylbenzimidazole-4-carboxamides including 2-(4-hydroxphenyl) benzimidazole 4-carboxamide, quinoxalinecarboxamides, imidazopyridinecarboxamides, 2-phenylindoles, 2-substituted benzoxazoles, such as 2-phenyl benzoxazole and 2-(3-methoxyphenyl) benzoxazole, 2-substituted benzimidazoles, such as 2-phenyl benzimidazole and 2-(3-methoxyphenyl) benzimidazole, 1,3,4,5 tetrahydro-azepino[5,4,3-cd]indol-6-one, azepinoindoles and azepinoindolones such as 1,5 dihydro-azepino[4,5,6-cd]indolin-6-one and dihydrodiazapinoindolinone, 3-substituted dihydrodiazapinoindolinones, such as 3-(4-trifluoromethylphenyl)-dihydrodiazapinoindolinone, tetrahydrodiazapinoindolinone and 5,6-dihydroimidazo[4,5,1-j,k][1,4]benzodiazopin-7(4H)-one, 2-phenyl-5,6-dihydro-imidazo[4,5,1-jk][1,4]benzodiazepin-7(4H)-one and 2,3, dihydro-isoindol-1-one, and analogues and derivatives thereof

5. Phthalazin-1(2H)-ones and quinazolinones, such as 4-hydroxyquinazoline, phthalazinone, 5-methoxy-4-methyl-1(2) phthalazinones, 4-substituted phthalazinones, 4-(1-piperazinyl)-1(2H)-phthalazinone, tetracyclic benzopyrano[4,3,2-de]phthalazinones and tetracyclic indeno[1,2,3-de]phthalazinones and 2-substituted quinazolines, such as 8-hydroxy-2-methylquinazolin-4-(3H) one, tricyclic phthalazinones and 2-aminophthalhydrazide, and analogues and derivatives thereof and 1(2H)-phthalazinone and derivatives thereof, as described in WO02/36576.

6. Isoindolinones and analogues and derivatives thereof

7. Phenanthridines and phenanthridinones, such as 5[H]phenanthridin-6-one, substituted 5[H]phenanthridin-6-ones, especially 2-, 3-substituted 5[H]phenanthridin-6-ones and sulfonamide/carbamide derivatives of 6(5H)phenanthridinones, thieno[2,3-c]isoquinolones such as 9-amino thieno[2,3-c]isoquinolone and 9-hydroxythieno[2,3-c]isoquinolone, 9-methoxythieno[2,3-c]isoquinolone, and N-(6-oxo-5,6-dihydrophenanthridin-2-yl]-2-(N,N-dimethylamino}acetamide, substituted 4,9-dihydrocyclopenta[lmn]phenanthridine-5-ones, and analogues and derivatives thereof.

8. Benzopyrones such as 1,2-benzopyrone, 6-nitrosobenzopyrone, 6-nitroso 1,2-benzopyrone, and 5-iodo-6-aminobenzopyrone, and analogues and derivatives thereof.

9. Unsaturated hydroximic acid derivatives such as 0-(3-piperidino-2-hydroxy-1-propyl)nicotinic amidoxime, and analogues and derivatives thereof.

10. Pyridazines, including fused pyridazines and analogues and derivatives thereof.

11. Other compounds such as caffeine, theophylline, and thymidine, and analogues and derivatives thereof.

Additional PARP inhibitors are described for example in U.S. Pat. Nos. 6,635,642, 5,587,384, WO2003080581, WO2003070707, WO2003055865, WO2003057145, WO2003051879, U.S. Pat. No. 6,514,983, WO2003007959, U.S. Pat. No. 6,426,415, WO2003007959, WO2002094790, WO2002068407, U.S. Pat. No. 6,476,048, WO2001090077, WO2001085687, WO2001085686, WO2001079184, WO2001057038, WO2001023390, WO2001021615, WO2001016136, WO2001012199, WO9524379, Banasik et al. J. Biol. Chem., 267:3, 1569-75 (1992), Banasik et al. Molec. Cell. Biochem. 138:185-97 (1994)), Cosi (2002) Expert Opin. Ther. Patents 12 (7), and Southan & Szabo (2003) Curr Med Chem 10 321-340 and references therein.

Preferred examples of PARP inhibitors that may be used in accordance with the present invention include Olaparib (AZD2281; 1-(Cyclopropylcarbonyl)-4-[5-[(3,4-dihydro-4-oxo-1-phthalazinyl)methyl]-2-fluorobenzoyl]piperazine; Pubchem CID 23725625), Rucaparib (AG014699; 8-Fluoro-2-{4-[(methylamino)methyl]phenyl}-1,3,4,5-tetrahydro-6H-azepino[5,4,3-cd]indol-6-one; pubchem CID 9931954), Niraparib (MK4827; 2-{4-[(3S)-3-Piperidinyl]phenyl}-2H-indazole-7-carboxamide; Pubchem CID CID: 24958200), Talazoparib (BMN-673; (8S,9R)-5-Fluoro-8-(4-fluorophenyl)-9-(1-methyl-1H-1,2,4-triazol-5-yl)-2,7,8,9-tetrahydro-3H-pyrido[4,3,2-de]phthalazin-3-one; Pubchem CID 135565082), Veliparib (ABT-888; 2-[(2R)-2-Methyl-2-pyrrolidinyl]-1H-benzimidazole-4-carboxamide; Pubchem CID 11960529), pamiparib (BGB-290; (10aR)-2-Fluoro-5,8,9,10,10a,11-hexahydro-10a-methyl-5,6,7a,11-tetraazacyclohepta[def]cyclopenta[a]fluoren-4(7H)-one; Pubchem CID: 135565554), CEP-9722 (11-methoxy-2-((4-methylpiperazin-1-yl)methyl)-4,5,6,7-tetrahydro-1H-cyclopenta[a]pyrrolo[3,4-c]carbazole-1,3(2H)-dione; Pubchem CID 24780387), E7016 (10-((4-Hydroxypiperidin-1-yl)methyl)chromeno[4,3,2-de]phthalazin-3(2H)-one; Pubchem CID 11660296), lobenguane (1-(3-iodobenzyl)guanidine; PubChem CID 60860), Cediranib (AZD-2171; Recentin; 4-[(4-fluoro-2-methyl-1H-indol-5-yl)oxy]-6-methoxy-7-[3-(pyrrolidin-1-yl)propoxy]quinazoline; PubChem CID 9933475), SH33162, 2x 121-2X, Ceralasertib (imino-methyl-[1-[6-[(3R)-3-methylmorpholin-4-yl]-2-(1H-pyrrolo[2,3-b]pyridin-4-yl)pyrimidin-4-yl]cyclopropyl]-oxo-λ⁶-sulfane; PubChem CID 54761306), JP1289 (Amelparib; 10-ethoxy-8-(morpholin-4-ylmethyl)-2,3,4,6-tetrahydro-1H-benzo[h][1,6]naphthyridin-5-one; PubChem CID 58424881), JP1547, RBN2397, IDX1197 (NOV1401), IMP4297 (1-(4-fluoro-3-(4-(pyrimidin-2-yl) piperazine-1-carbonyl)benzyl)quinazoline-2,4(1H,3H)5-fluoro-dione), SC10914, HWH340, SOMCL9112 (4-(4-fluoro-3-(5-methyl-3-(trifluoromethyl)-5,6,7,8-4H-[1,2,4]triazolo[4,3-a]piperazine-7-carbonyl)benzyl)phthalazin-1(2H)-one) ABT767; WB1340; and STX-100s.

In some preferred embodiments, the PARP inhibitor may be olaparib.

In other preferred embodiments, the PARP inhibitor may be rucaparib.

In other preferred embodiments, the PARP inhibitor may be niraparib.

In other preferred embodiments, the PARP inhibitor may be talazoparib.

In other preferred embodiments, the PARP inhibitor may be velaparib.

Deficiencies in 2′-deoxynucleoside 5′-phosphate N-hydrolase 1 are shown herein to sensitise HR deficient cells to treatment with a PARP inhibitor. For example, the potency of the PARP inhibitor may be increased when the activity of DNPH1 in the HR deficient cells is reduced. In some embodiments, the potency of the PARP inhibitor as measured by IC50 may be increased by 2 fold or more. This may be useful, for example in increasing the efficacy of a PARP inhibitor for the treatment of HR deficient cancer or reducing the dose of the PARP inhibitor that is required to elicit an anti-cancer effect in an HR deficient cancer and hence reducing toxicity in patients.

2′-deoxynucleoside 5′-phosphate N-hydrolase 1 (DNPH1; Gene ID 10591) is glycohydrolase that cleaves the N-glycosidic bond of deoxyribonucleoside 5′-phosphates. DNPH1 is a c-myc stimulated transcription factor that participates in the regulation of cell proliferation, differentiation, and apoptosis. DNPH1 may have the reference amino acid sequence of NP_006434.1 or NP_954653.1 ora variant thereof and may be encoded by the nucleotide sequence of NM_006443.3 or NM_199184.2 or a variant thereof.

Reducing DNPH1 activity as described herein may cause DNPH1 to be completely inactivated (i.e. DNPH1 activity may be reduced to zero or substantially zero), or reduced by 50% or more, 60% or more, 70% or more, 80% or more, 90% or more or 95% or more in the HR deficient cells relative to cells in which DNPH1 is not reduced.

DNPH1 activity may be reduced systemically in the individual (i.e. all the cells of the individual may be affected). Alternatively, DNPH1 activity may be reduced selectively (i.e. only certain types of cells of the individual may be affected). For example, DNPH1 activity may be selectively reduced in cancer cells, stromal cells, or endothelial cells of the individual. Selective reduction of DNPH1 activity may be achieved by the direct administration of an agent which reduces DNPH1 activity to target cells, such as a tumour e.g. by injection. Selective reduction of DNPH1 activity may be achieved using conventional techniques, such as cell targeted delivery vehicles, such as viral vectors that express a ligand for a specific cell type.

In some embodiments, DNPH1 activity may be reduced by administering an agent that reduces or inhibits DNPH1 activity, such as a DNPH1 antagonist. DNPH1 antagonists may include any agent capable of antagonising, inhibiting, blocking or down-regulating DNPH1.

Suitable agents for reducing DNPH1 activity may include DNPH1 inhibitors. DNPH1 inhibitors may, for example, include small chemical molecules, for example non-polymeric organic compounds having a molecular weight of 900 Daltons or less.

Suitable DNPH1 inhibitors may include 2′-deoxynucleoside 5′-phosphate analogues and derivatives or pro-forms of such compounds, for example cell-permeable pro-forms. For example, the DNPH1 inhibitor may comprise an N6-substituted AMP, such as N6BA (N6-benzyladenosine), N6-isopentenyladenosine, or N6-furfuryladenosine, or a 6-aryl- or 6-heteroarylpurine riboside 5′-monophosphate, or a pharmaceutically acceptable salt, solvate, or derivative thereof.

Other suitable DNPH1 inhibitors are also known in the art (Amiable et al 2013 Plos One 8 11 e8075; Amiable et al Eur J Med Chem. 2014 Oct. 6; 85:418-37).

Suitable assays for measuring DNPH1 inhibition are known in the art. DNPH1 activity may, for example, be determined spectrophotometrically by incubating DNPH1 with dGMP and by following the production of 2-deoxyribose 5-phosphate (Dupouy et al (2010) J. Biol. Chem. 285 53 41806-41814).

The terms “DNPH1 antagonist” and “DNPH1 inhibitor” as used herein, cover pharmaceutically acceptable salts and solvates of these compounds.

Techniques for the rational design of small molecule inhibitors through structural analysis of target proteins are well-known in the art.

Suitable agents for reducing DNPH1 activity may also include suppressor nucleic acids, targetable nucleases and nucleic acids encoding such agents.

These agents may reduce DNPH1 activity in a cell by down-regulating production or reducing expression of active DNPH1 polypeptide. The use of nucleic acid suppression and targetable nucleases to down regulate the expression of a target gene is well known in the art and described in more detail below.

In some embodiments, expression of DNPH1 may be reduced or prevented using suppressor nucleic acids through anti-sense or RNAi technology. The use of these approaches to down-regulate gene expression is now well-established in the art.

Anti-sense oligonucleotides may be designed to hybridise to the complementary sequence of nucleic acid, pre-mRNA or mature mRNA, interfering with the production of the base excision repair pathway component so that its expression is reduced or completely or substantially completely prevented. In addition to targeting coding sequence, anti-sense techniques may be used to target control sequences of a gene, e.g. in the 5′ flanking sequence, whereby the anti-sense oligonucleotides can interfere with expression control sequences. The construction of anti-sense sequences and their use is described for example in Peyman & Ulman, Chemical Reviews, 90:543-584, 1990 and Crooke, Ann. Rev. Pharmacol. Toxicol., 32:329-376, 1992.

Suppressor oligonucleotides may be generated in vitro or ex vivo for administration or anti-sense RNA may be generated in vivo within cells in which down-regulation is desired. Thus, double-stranded DNA may be placed under the control of a promoter in a “reverse orientation” such that transcription of the anti-sense strand of the DNA yields RNA which is complementary to normal mRNA transcribed from the sense strand of the target gene. The complementary anti-sense RNA sequence is thought then to bind with mRNA to form a duplex, inhibiting translation of the endogenous mRNA from the target gene into protein. Whether or not this is the actual mode of action is still uncertain. However, it is established fact that the technique works.

The complete sequence corresponding to the coding sequence in reverse orientation need not be used. For example fragments of sufficient length may be used. It is a routine matter for the person skilled in the art to screen fragments of various sizes and from various parts of the coding or flanking sequences of a gene to optimise the level of anti-sense inhibition. It may be advantageous to include the initiating methionine ATG codon, and perhaps one or more nucleotides upstream of the initiating codon. A suitable fragment may have about 14-23 nucleotides, e.g., about 15, 16 or 17 nucleotides. For example, a suitable suppressor nucleic acid may comprise a nucleotide sequence having a contiguous sequence of about 14-23 nucleotides of SEQ ID NO: 2 or a variant thereof.

An alternative to anti-sense is to use a copy of all or part of the target gene inserted in sense, which is the same orientation as the target gene, to achieve reduction in expression of the target gene by co-suppression (Angell & Baulcombe, The EMBO Journal 16(12):3675-3684, 1997 and Voinnet & Baulcombe, Nature, 389: 553, 1997). Double stranded RNA (dsRNA) has been found to be even more effective in gene silencing than either sense or antisense strands alone (Fire et al, Nature 391, 806-811, 1998). dsRNA mediated silencing is gene specific and is often termed RNA interference (RNAi). Methods relating to the use of RNAi to silence genes in C. elegans, Drosophila, plants, and mammals are known in the art (Fire, Trends Genet., 15: 358-363, 19999; Sharp, RNA interference, Genes Dev. 15: 485-490 2001; Hammond et al., Nature Rev. Genet. 2: 110-1119, 2001; Tuschl, Chem. Biochem. 2: 239-245, 2001; Hamilton et al., Science 286: 950-952, 1999; Hammond, et al., Nature 404: 293-296, 2000; Zamore et al., Cell, 101: 25-33, 2000; Bernstein, Nature, 409: 363-366, 2001; Elbashir et al, Genes Dev., 15: 188-200, 2001; WO01/29058; WO99/32619, and Elbashir et al, Nature, 411: 494-498, 2001).

RNA interference is a two-step process. First, dsRNA is cleaved within the cell to yield short interfering RNAs (siRNAs) of about 21-23 nt length with 5′ terminal phosphate and 3′ short overhangs (˜2 nt). The siRNAs target the corresponding mRNA sequence specifically for destruction (Zamore, Nature Structural Biology, 8, 9, 746-750, 2001.

RNAi may also be efficiently induced using chemically synthesized siRNA duplexes of the same structure with 3′-overhang ends (Zamore et al, Cell, 101: 25-33, 2000). Synthetic siRNA duplexes have been shown to specifically suppress expression of endogenous and heterologous genes in a wide range of mammalian cell lines (Elbashir et al, Nature, 411: 494-498, 2001).

Another possibility is that nucleic acid is used which on transcription produces a ribozyme, able to cut nucleic acid at a specific site and therefore also useful in influencing gene expression, e.g., see Kashani-Sabet & Scanlon, Cancer Gene Therapy, 2(3): 213-223, 1995 and Mercola & Cohen, Cancer Gene Therapy, 2(1): 47-59, 1995.

Small RNA molecules may be employed to regulate gene expression. These include targeted degradation of mRNAs by small interfering RNAs (siRNAs), post transcriptional gene silencing (PTGs), developmentally regulated sequence-specific translational repression of mRNA by micro-RNAs (miRNAs), and targeted transcriptional gene silencing.

A role for the RNAi machinery and small RNAs in targeting of heterochromatin complexes and epigenetic gene silencing at specific chromosomal loci has also been demonstrated. Double-stranded RNA (dsRNA)-dependent post transcriptional silencing, also known as RNA interference (RNAi), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response. The decrease in expression of targeted gene products can be extensive with 90% silencing induced by a few molecules of siRNA.

For example, a suitable suppressor nucleic acid may comprise a nucleotide sequence having a contiguous sequence of 10 to 30 nucleotides of SEQ ID NO: 2 or a variant thereof, for example 15 to 25 nucleotides.

In the art, these RNA sequences are termed “short or small interfering RNAs” (siRNAs) or “microRNAs” (miRNAs) depending on their origin. Both types of sequence may be used to down-regulate gene expression by binding to complementary RNAs and either triggering mRNA elimination (RNAi) or arresting mRNA translation into protein. siRNA are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

The siRNA ligands are typically double stranded and, in order to optimise the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNA ligands are typically single stranded and have regions that are partially complementary enabling the ligands to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is discussed in John et al, PLoS Biology, 11(2), 1862-1879, 2004.

Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. For example, a suppressor may have between 10 and 40 contiguous ribonucleotides (or synthetic analogues thereof), more preferably between 17 and 30 contiguous ribonucleotides, more preferably between 19 and 25 contiguous ribonucleotides and most preferably between 21 and 23 contiguous ribonucleotides of SEQ ID NO: 2 or a variant thereof. In some embodiments of the invention employing double-stranded siRNA, the molecule may have symmetric 3′ overhangs, e.g. of one or two (ribo)nucleotides, typically a UU of dTdT 3′ overhang. Based on the disclosure provided herein, the skilled person can readily design suitable siRNA and miRNA sequences, for example using resources such as Ambion's siRNA finder (available on-line). siRNA and miRNA sequences can be synthetically produced and added exogenously to cause gene downregulation or produced using expression systems (e.g. vectors). In a preferred embodiment the siRNA is synthesized synthetically.

Longer double stranded RNAs may be processed in the cell to produce siRNAs (e.g. see Myers, Nature Biotechnology, 21: 324-328, 2003). The longer dsRNA molecule may have symmetric 3′ or 5′ overhangs, e.g. of one or two (ribo)nucleotides, or may have blunt ends. The longer dsRNA molecules may be 25 nucleotides or longer. For example, 25 or more contiguous nucleotides of SEQ ID NO: 2. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length. dsRNAs 30 nucleotides or more in length may be expressed using the vector pDECAP (Shinagawa et al., Genes and Dev., 17: 1340-5, 2003). For example, a suppressor nucleic acid may have between 25 and 30 contiguous nucleotides (or synthetic analogues thereof), more preferably between 25 and 27 contiguous nucleotides, more preferably 27 contiguous nucleotides of SEQ ID NO: 2 or a variant thereof,

Another alternative is the expression of a short hairpin RNA molecule (shRNA) in the cell. shRNAs are more stable than synthetic siRNAs. A shRNA consists of short inverted repeats separated by a small loop sequence. One inverted repeat is complimentary to the gene target. In the cell the shRNA is processed by DICER into a siRNA which degrades the target gene mRNA and suppresses expression. In a preferred embodiment the shRNA is produced endogenously (within a cell) by transcription from a vector. shRNAs may be produced within a cell by transfecting the cell with a vector encoding the shRNA sequence under control of a RNA polymerase III promoter such as the human H1 or 7SK promoter or a RNA polymerase II promoter. Alternatively, the shRNA may be synthesised exogenously (in vitro) by transcription from a vector. The shRNA may then be introduced directly into the cell. Preferably, the shRNA sequence is between 40 and 100 bases in length, more preferably between 40 and 70 bases in length. The stem of the hairpin is preferably between 19 and 30 base pairs in length. The stem may contain G-U pairings to stabilise the hairpin structure.

Nucleic acid encoding a suppressor nucleic acid may be contained in a vector. Suitable expression vectors are well-known in the art and include viral vectors, such as retroviral, adenoviral, adeno-associated viral, lentiviral, vaccinia or herpes vectors.

In some embodiments, the suppressor nucleic acid, such as siRNA, longer dsRNA or miRNA, is produced endogenously (within a cell) by transcription from the vector. The vector may be introduced into the cell in any of the ways known in the art. Optionally, expression of the RNA sequence can be regulated using a tissue specific promoter. In other embodiments, the suppressor nucleic acid, such as siRNA, longer dsRNA or miRNA, is produced exogenously (in vitro) by transcription from a vector. Cells may be transfected with the suppressor nucleic acid (i.e. a nucleic acid molecule which suppresses DNPH1 expression), such as an siRNA or shRNA, or a heterologous nucleic acid or vector encoding the suppressor nucleic acid.

Alternatively, suppressor nucleic acid, such as siRNA molecules may be synthesized using standard solid or solution phase synthesis techniques, which are known in the art. Linkages between nucleotides may be phosphodiester bonds or alternatives, e.g., linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through —O— or —S—.

Modified nucleotide bases can be used in addition to the naturally occurring bases, and may confer advantageous properties on siRNA molecules containing them. For example, modified bases may increase the stability of the siRNA molecule, thereby reducing the amount required for silencing. The provision of modified bases may also provide siRNA molecules, which are more, or less, stable than unmodified siRNA.

The term ‘modified nucleotide base’ encompasses nucleotides with a covalently modified base and/or sugar. For example, modified nucleotides include nucleotides having sugars, which are covalently attached to low molecular weight organic groups other than a hydroxyl group at the 3′ position and other than a phosphate group at the 5′ position. Thus, modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2; azido-ribose, carbocyclic sugar analogues a-anomeric sugars; epimeric sugars such as arabinose, xyloses or lyxoses, pyranose sugars, furanose sugars and sedoheptulose.

Modified nucleotides are known in the art and include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N6-methyladenine, 4-acetylcytosine, 5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5methoxyuracil, 2 methylthio-N6-isopentenyladenine, uracil-5-oxyacetic acid methyl ester, psueouracil, 2-thiocytosine, 5-methyl-2 thiouracil, 2-thiouracil, 4-thiouracil, 5methyluracil, N-uracil-5-oxyacetic acid methylester, uracil 5-oxyacetic acid, queosine, 2-thiocytosine, 5-propyluracil, 5-propylcytosine, 5-ethyluracil, 5ethylcytosine, 5-butyluracil, 5-pentyluracil, 5-pentylcytosine, and 2,6,diaminopurine, methylpsuedouracil, 1-methylguanine, 1-methylcytosine.

In other embodiments, the activity of DNPH1 may be reduced using targeted mutagenesis to reduce expression of active DNPH1 polypeptide to the individual. For example, targeted mutagenesis may introduce a causing a deletion, insertion or frameshift at the target sequence of the DNPH1 gene which reduces or blocks expression of active DNPH1 polypeptide. The use of targeted mutagenesis techniques such as gene editing with targeted nucleases, to knock out or abolish expression of target genes is well-established in the art (see for example Gaj et al (2013) Trends Biotechnol. 31(7) 397-405).

Targeted mutagenesis to introduce one or more mutations may be performed by any convenient method. For example, cells may be transfected with a heterologous nucleic acid which encodes a targetable nuclease. The targetable nuclease may inactivate the DNPH1 gene encoding DNPH1 in one or more cells of the individual, for example, by introducing one or more mutations that prevent the expression of active DNPH1 polypeptide.

The heterologous nucleic acid may include an inducible promoter that promotes expression of the targetable nuclease and optional targeting sequence within a specific cell type, for example a tumour cell. For example, the inducible promoter could be a promoter-enhancer cassette that selectively favours expression of the targetable nuclease and the optional targeting sequence within the tumour cell over other types of host cells.

Suitable targetable nucleases include, for example, site-specific nucleases, such as zinc-finger nucleases (ZFNs), transcription activator-like effector nucleases (TALENs), and meganucleases or RNA guided nucleases, such as clustered regularly interspaced short palindromic repeat (CRISPR) nucleases, which may be administered in combination with a guide RNA that recognises a target sequence within the DNPH1 gene.

Zinc-finger nucleases (ZFNs) comprise one or more Cys₂-His₂ zinc-finger DNA binding domains and a cleavage domain (i.e., nuclease). The DNA binding domain may be engineered to recognize and bind to any nucleic acid sequence using conventional techniques (see for example Qu et al. (2013) Nucl Ac Res 41(16):7771-7782). The use of ZFNs to introduce mutations into target genes is well-known in the art (see for example, Beerli et al Nat. Biotechnol. 2002; 20:135-141; Maeder et al Mol. Cell. 2008; 31:294-301; Gupta et al Nat. Methods. 2012; 9:588-590) and engineered ZFNs are commercially available (Sigma-Aldrich (St. Louis, Mo.).

Transcription activator-like effector nucleases (TALENs) comprise a nonspecific DNA-cleaving nuclease fused to a DNA-binding domain comprising a series of modular TALE repeats linked together to recognise a contiguous nucleotide sequence. The use of TALEN targeting nucleases is well known in the art (e.g. Joung & Sander (2013) Nat Rev Mol Cell Bio 14:49-55; Kim et al Nat Biotechnol. (2013); 31:251-258. Miller J C, et al. Nat. Biotechnol. (2011) 29:143-148. Reyon D, et al. Nat. Biotechnol. (2012); 30:460-465).

Meganucleases are endodeoxyribonucleases characterized by a large recognition site (double-stranded DNA sequences of 12 to 40 base pairs); as a result this site generally occurs only once in any given genome (see for example Silva et al. (2011) Curr Gene Ther 11(1):11-27).

CRISPR targeting nucleases (e.g. Cas9) complex with a guide RNA (gRNA) to cleave genomic DNA in a sequence-specific manner. The crRNA and tracrRNA of the guide RNA may be used separately or may be combined into a single RNA to enable site-specific mammalian genome cutting within the DNPH1 gene or its regulatory elements. The use of CRISPR/Cas9 systems to introduce insertions or deletions into genes as a way of decreasing transcription is well known in the art (see for example Cader et al Nat Immunol 2016 17 (9) 1046-1056, Hwang et al. (2013) Nat. Biotechnol 31:227-229; Xiao et al., (2013) Nucl Acids Res 1-11; Horvath et al., Science (2010) 327:167-170; Jinek M et al. Science (2012) 337:816-821; Cong L et al. Science (2013) 339:819-823; Jinek M et al. (2013) eLife 2:e00471; Mali P et al. (2013) Science 339:823-826; Qi L S et al. (2013) Cell 152:1173-1183; Gilbert L A et al. (2013) Cell 154:442-451; Yang H et al. (2013) Cell 154:1370-1379; and Wang H et al. (2013) Cell 153:910-918).

In some preferred embodiments, the targetable nuclease is a Cas endonuclease, preferably Cas9, which is expressed in the immune cells in combination with a guide RNA targeting sequence that targets the Cas endonuclease to cleave genomic DNA within the DNPH1 gene and generate insertions or deletions that prevent expression of active DNPH1 polypeptide.

Nucleic acid sequences encoding a suppressor nucleic acid or targetable nuclease and optionally a guide RNA may be comprised within an expression vector. Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the encoding nucleic acid in a host cell. Suitable regulatory sequences to drive the expression of heterologous nucleic acid coding sequences in a range of expression systems are well-known in the art and include constitutive promoters, for example viral promoters such as CMV or SV40. A vector may also comprise sequences, such as origins of replication and selectable markers, which allow for its selection and replication and expression in bacterial hosts, such as E. coli and/or in eukaryotic cells, such as yeast, insect or mammalian cells. Vectors suitable for use in expressing a suppressor nucleic acid or targetable nuclease in mammalian cells include plasmids and viral vectors e.g. retroviruses, lentiviruses, adenoviruses, and adeno-associated viruses. Suitable techniques for expressing a suppressor nucleic acid or targetable nuclease in mammalian cells are well known in the art (see for example; Molecular Cloning: a Laboratory Manual: 3rd edition, Russell et al., 2001, Cold Spring Harbor Laboratory Press or Protocols in Molecular Biology, Second Edition, Ausubel et al. eds. John Wiley & Sons, 1992; Recombinant Gene Expression Protocols Ed RS Tuan (March 1997) Humana Press Inc).

Transfection with the vector or nucleic acid may be stable or transient. Suitable techniques for transfecting immune cells are well known in the art.

In addition to increasing the potency of PARP inhibition, reduced DNPH1 activity is shown herein to sensitive HR deficient cancer cells to treatment with 5-modified-2′-deoxypyrimidine nucleosides. In addition, treatment of HR deficient cancer cells with 5-modified-2′-deoxypyrimidine nucleosides is shown to increase the potency of PARP inhibition of the cancer cells.

A 5-modified-2′-deoxypyrimidine nucleoside may include 5-modified-2′-deoxyuridine nucleosides, such as 5-hydroxymethyl-2′-deoxyuridine and 5-formyl-2′-deoxyuridine, and 5-modified-2′-deoxycytidine nucleosides, such as 5-hydroxymethyl-2′-deoxycytidine and 5-formyl-2′-deoxycytidine.

Suitable 5-modified-2′-deoxypyrimidine nucleosides include nucleosides that are substrates of SMUG1 when incorporated into a DNA strand.

5-modified-2′-deoxpyrimidine nucleosides may be synthesised using standard chemical synthesis techniques or obtained from commercial suppliers (e.g. Sigma-Aldrich).

Inactivation of SMUG1 is shown herein to promote resistance to treatment with combinations of PARP inhibitors and 5-modified-2′-deoxypyrimidine nucleosides. Reducing the activity of SMUG1 in an organ or tissue may therefore be useful in reducing the toxicity of the combination treatment in the tissue or organ.

Single-strand selective monofunctional uracil DNA glycosylase (SMUG1; Gene ID 23583) is a uracil-DNA glycosylase that excises uracil from single and double stranded DNA. DNPH1 may have the reference amino acid sequence of NP_001230716.1, NP_001230717.1, NP_001230718.1, NP_001230719.1, or NP_001230720.1 or a variant thereof and may be encoded by the nucleotide sequence of NM_001243787.1, NM_001243788.1, NM_001243789.2, NM_001243790.2, or a NM_001243791.2 or a variant thereof

Toxicity in a tissue or organ of an individual undergoing treatment with a combination of a PARP inhibitor and a 5-modified-2′-deoxypyrimidine nucleoside may be reduced or ameliorated by selectively reducing SMUG1 activity in the tissue or organ of the individual.

Toxicity may be reduced or ameliorated in any non-cancerous tissue or organ in which toxicity occurs following treatment with a PARP inhibitor. Suitable tissues or organs may include bone marrow, kidneys, intestines and hair follicles (LaFargue et al Lancet Oncol 2019 20(1) e15-e28).

Suitable delivery systems to reduce SMUG1 activity selectively in an organ or tissue include macromolecular carriers, such as liposomes and nanoparticles, are well-known in the art (Mu et al Biomaterials (2018) 155 191-202; Zhou et al Acta Pharm Sin B (2014) 4 (1) 37-42).

In some embodiments, SMUG1 activity may be reduced by administering an agent that reduces or inhibits SMUG1 activity, such as a SMUG1 antagonist. A SMUG1 antagonist may include any agent capable of antagonising, inhibiting, blocking or down-regulating SMUG1.

SMUG1 antagonists may include SMUG1 inhibitors. SMUG1 inhibitors may, for example, include small chemical molecules, for example non-polymeric organic compounds having a molecular weight of 900 Daltons or less. SMUG1 antagonists may also include suppressor nucleic acids, targetable nucleases and nucleic acids encoding such agents. Suppressor nucleic acids and targetable nucleases are described in more detail above.

An individual undergoing treatment with a combination of a PARP inhibitor and a 5-modified-2′-deoxypyrimidine nucleoside may have an HR deficient cancer.

An active agent described herein, such as a PARP inhibitor, 5 modified-2′-deoxypyrimidine nucleoside, DNPH1 antagonist, SMUG1 antagonist, suppressor nucleic acid, targetable nuclease, nucleic acid encoding a suppressor nucleic acid or targetable nuclease, may be administered alone or more usually in the form of a pharmaceutical composition, which may comprise at least one component in addition to the active agent. The active agent may be admixed with other reagents, such as buffers, carriers, diluents, preservatives and/or pharmaceutically acceptable excipients in order to produce a pharmaceutical composition for use in cancer immunotherapy. Suitable reagents are described in more detail below.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Pharmaceutical compositions suitable for administration (e.g. by infusion), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents. Examples of suitable isotonic vehicles for use in such formulations include Sodium Chloride Injection, Ringer's Solution, or Lactated Ringer's Injection. Suitable vehicles can be found in standard pharmaceutical texts, for example, Remington's Pharmaceutical Sciences, 18th edition, Mack Publishing Company, Easton, Pa., 1990.

An active agent or pharmaceutical composition as described herein may be administered to a subject by any convenient route of administration, whether systemically/peripherally or at the site of desired action, including but not limited to; oral or parenteral, for example, by injection or infusion, for example intravenous infusion. Suitable administration techniques are known in the art and commonly used in therapy (see, e.g., Rosenberg et al., New Eng. J. of Med., 319:1676, 1988).

It will be appreciated that appropriate dosages of the active agent, and compositions comprising the active agent, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular cells, the route of administration, the time of administration, the rate of loss or inactivation of the cells, the duration of the treatment, other drugs, compounds, and/or materials used in combination, and the age, sex, weight, condition, general health, and prior medical history of the patient. The amount of cells and the route of administration will ultimately be at the discretion of the physician, although generally the dosage will be to achieve local concentrations at the site of action which achieve the desired effect without causing substantial harmful or deleterious side-effects.

A typical oral dosage of an active agent, such as a small molecule inhibitor, is in the range of from about 0.05 to about 1000 mg, preferably from about 0.1 to about 500 mg, and more preferred from about 1.0 mg to about 200 mg administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art. For parenteral routes such as intravenous, intrathecal, intramuscular and similar administration, typically doses are in the order of about half the dose employed for oral administration.

An active agent, such as a PARP inhibitor, 5 modified-2′-deoxypyrimidine nucleoside, DNPH1 antagonist, SMUG1 antagonist, suppressor nucleic acid, targetable nuclease, nucleic acid encoding a suppressor nucleic acid or targetable nuclease, may be useful in therapy, as described herein. For example, an active agent which reduces DNPH1 activity may be administered to an individual for the treatment of HR deficient cancer in combination with a PARP inhibitor and/or 5 modified-2′-deoxypyrimidine nucleoside. A PARP inhibitor may be administered to an individual for the treatment of HR deficient cancer in combination with a 5 modified-2′-deoxypyrimidine nucleoside.

Cancer is characterised by the abnormal proliferation of malignant cancer cells relative to normal cells and may include leukaemia, such as AML, CML, ALL and CLL, lymphoma, such as Hodgkin lymphoma, non-Hodgkin lymphoma and multiple myeloma, and solid cancers such as sarcomas, skin cancer, melanoma, bladder cancer, brain cancer, breast cancer, uterus cancer, oral cancer, ovary cancer, prostate cancer, lung cancer, colorectal cancer, cervical cancer, liver cancer, head and neck cancer, oesophageal cancer, pancreas cancer, renal cancer, adrenal cancer, stomach cancer, testicular cancer, cancer of the gall bladder and biliary tracts, thyroid cancer, thymus cancer, cancer of bone, and cerebral cancer. In some preferred embodiments, the cancer condition may be breast, ovary, pancreas or prostate cancer. Cancers may be familial or sporadic.

A HR deficient cancer is a cancer which is deficient in HR dependent DNA DSB repair. An HR deficient cancer may comprise or consist of cancer cells which have a reduced or abrogated ability to repair DNA DSBs by homologous recombination (HR) relative to normal cells i.e. the HR is dysfunctional in the cancer cells and the ability of the cancer cells to repair DNA DSBs using HR is reduced or abolished.

The activity of one or more proteins that mediate the repair of DNA DSBs by HR (i.e. HR proteins) may be reduced or abolished in the cancer cells of an individual having an HR deficient cancer. Proteins that mediate the repair of DNA DSBs by HR are well characterised in the art (see for example, Wood et al (2001) Science 291 1284-1289) and may include BRCA1, BRCA2, MUS81, RAD52, RAD51C, RAD50, ATM/ATR, FANC, BARD1, BRIP1, CHEK1, CHEK2, FAM175A, NBN, PALB2, MRE11A, NBS1, RBBP8 (CtIP), MRE11, RPA, MMR, H2AX, EME1 and TP53 and Fanconi anaemia (FA) proteins such as FANCA, FANCB, FANCC, FAND2, FANCE, FANCF, FANCG and FANCI.

One or more genes encoding a protein that mediates the repair of DNA DSBs by HR (i.e. an HR gene) may be mutated in the cancer cells of an individual having an HR deficient cancer. Mutations in one or more HR genes may reduce or abolish the expression or activity of an HR protein and thereby reduce or abolish HR activity in the cancer cells. HR genes may include BRCA1, BRCA2, MUS81, RAD52, RAD51C, RAD50, ATM/ATR, FANC, BARD1, BRIP1, CHEK1, CHEK2, FAM175A, NBN, PALB2, MRE11A, NBS1, RBBP8 (CtIP), MRE11, RPA, MMR, H2AX, EME1 and TP53 and Fanconi anaemia (FA) genes such as FANCA, FANCB, FANCC, FAND2, FANCE, FANCF, FANCG and FANCI.

In some preferred embodiments, the cancer cells may have a BRCA1 and/or a BRCA2 deficient phenotype i.e. BRCA1 and/or BRCA2 activity is reduced or abolished in the cancer cells. Cancer cells with this phenotype may be deficient in BRCA1 and/or BRCA2 i.e. expression and/or activity of BRCA1 and/or BRCA2 may be reduced or abolished in the cancer cells, for example by means of mutation or polymorphism in the encoding nucleic acid, or by means of mutation or polymorphism in a gene encoding a regulatory factor, for example the EMSY gene which encodes a BRCA2 regulatory factor (Hughes-Davies et al, Cell, Vol 115, pp 523-535). BRCA1 and BRCA2 are known tumour suppressors whose wild-type alleles are frequently lost in tumours of heterozygous carriers (Jasin M. Oncogene. 2002 Dec. 16; 21(58):8981-93; Tutt et al Trends Mol Med. (2002)8(12):571-6). The association of BRCA1 and/or BRCA2 mutations with breast cancer is well-characterised in the art (Radice P J Exp Clin Cancer Res. 2002 September; 21(3 Suppl):9-12). Amplification of the EMSY gene, which encodes a BRCA2 binding factor, is also known to be associated with breast and ovarian cancer. Carriers of mutations in BRCA1 and/or BRCA2 are also at elevated risk of cancer of the ovary, prostate and pancreas.

In some embodiments, an HR deficient cancer that has developed resistance to PARP inhibition may be treated as described herein. A PARP inhibition resistant HR deficient cancer may be deficient in PARP1, PARG deficient or one or more components of the TP53BP1 pathway. In some embodiments, HR may be re-activated in the HR deficient cancer, for example through inactivation of the p53 binding protein 1 (TP53BP1) pathway. Treatment with a 5-modified-2′-deoxpyrimidine nucleoside in combination with an agent that reduces DNPH1 activity is shown herein to exert a cytotoxic effect on HR deficient cancer cells that have developed PARP inhibition resistance.

Poly(ADP-ribose) glycohydrolase (PARG; Gene ID 8505) catabolizes poly(ADP-ribose) in cells. PARG may have the amino acid sequence of database accession number NP_001290415.1 or a variant thereof and may be encoded by a nucleotide of database accession number NM_001303486.2 or a variant thereof.

p53 binding protein 1 (TP53BP1; Gene ID 7158) is involved in the DNA damage response and DNA repair. TP53BP1 may have the amino acid sequence of database accession number NP_001135451.1 or a variant thereof and may be encoded by a nucleotide of database accession number NM_001141979.1 or a variant thereof.

In some embodiments, a cancer in an individual may have been previously identified as being HR deficient. In other embodiments, a method as described herein may comprise the step of identifying a cancer in an individual as HR deficient. Suitable methods of identifying an HR deficient cancer are well known in the art.

An individual suitable for treatment as described herein, may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-utan, gibbon), or a human. In some preferred embodiments, the individual is a human. In other preferred embodiments, non-human mammals, especially mammals that are conventionally used as models for demonstrating therapeutic efficacy in humans (e.g. murine, primate, porcine, canine, or rabbit animals) may be employed.

In some embodiments, the individual may have minimal residual disease (MRD) after an initial cancer treatment.

An individual with cancer may display at least one identifiable sign, symptom, or laboratory finding that is sufficient to make a diagnosis of cancer in accordance with clinical standards known in the art. Examples of such clinical standards can be found in textbooks of medicine such as Harrison's Principles of Internal Medicine, 15th Ed., Fauci A S et al., eds., McGraw-Hill, New York, 2001. In some instances, a diagnosis of a cancer in an individual may include identification of a particular cell type (e.g. a cancer cell) in a sample of a body fluid or tissue obtained from the individual.

The term “treatment”, as used herein in the context of treating a condition, pertains generally to treatment and therapy in which some desired therapeutic effect is achieved, for example, the inhibition of the progress of the condition, and includes a reduction in the rate of progress, a halt in the rate of progress and amelioration of the condition, and cure of the condition.

Treatment may be any treatment and therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment as a prophylactic measure (i.e. prophylaxis) is also included. For example, an individual susceptible to or at risk of the occurrence or re-occurrence of cancer may be treated as described herein. Such treatment may prevent or delay the occurrence or re-occurrence of cancer in the individual.

In particular, treatment may include inhibiting cancer growth, including complete cancer remission, and/or inhibiting cancer metastasis. Cancer growth generally refers to any one of a number of indices that indicate change within the cancer to a more developed form. Thus, indices for measuring an inhibition of cancer growth include a decrease in cancer cell survival, a decrease in tumor volume or morphology (for example, as determined using computed tomographic (CT), sonography, or other imaging method), a delayed tumor growth, a destruction of tumor vasculature, or improved performance in delayed hypersensitivity skin test.

Combinations of active agents as described herein, such as (i) a PARP inhibitor in combination with an agent that reduces DNPH1 activity; (ii) a PARP inhibitor in combination with a 5-modified-2′-deoxypyrimidine nucleoside; (iii) an agent that reduces DNPH1 activity in combination with 5-modified-2′-deoxypyrimidine nucleoside; or (iv) a PARP inhibitor, in combination with an agent that reduces DNPH1 activity and a 5-modified-2′-deoxypyrimidine nucleoside, may be administered in combination with one or more other therapies, such as cytotoxic chemotherapy or radiotherapy.

For example, a combination of active agents as described herein may be administered in combination with an anti-cancer agent. Examples of suitable anti-cancer agents include chemoactive agents, for example alkylating agents such as platinum complexes including cisplatin, mono(platinum), bis(platinum), tri-nuclear platinum complexes and carboplatin, thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU) gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, pitavastatin, fludarabine phosphate, and cladribine; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; binblastine; vindesine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); Topoisomerase inhibitors such as doxorubicin HCl, daunorubicin citrate, mitoxantrone HCl, actinomycin D, etoposide, topotecan HCl, teniposide (VM-26), and irinotecan and pharmaceutically acceptable salts, acids or derivatives of any of the above.

When the active agents are used in combination with additional active agents, the compounds may be administered either sequentially or simultaneously by any convenient route. When an active agent is used in combination with an additional active agent active against the same disease, the dose of each agent in the combination may differ from that when the active agents are used alone. Appropriate doses will be readily appreciated by those skilled in the art. The one or more additional active agents may be administered by any convenient means.

Administration of combinations of active agents as described herein, such as (i) a PARP inhibitor in combination with an agent that reduces DNPH1 activity; (ii) a PARP inhibitor in combination with 5-modified-2′-deoxypyrimidine nucleoside, (iii) an agent that reduces DNPH1 activity in combination with a 5-modified-2′-deoxypyrimidine nucleoside or (iv) a PARP inhibitor, in combination with an agent that reduces DNPH1 activity and a 5-modified-2′-deoxypyrimidine nucleoside, as described herein, can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

Other aspects of the invention relate to methods of screening to identify compounds as candidate compounds for use in the treatment of HR deficient cancer.

In some embodiments, a method of screening for a compound useful in the treatment of an HR cancer in combination with a PARP inhibitor or a 5-modified-2′-deoxypyrimidine nucleoside, may comprise; determining the activity of an isolated DNPH1 protein in the presence and absence of a test compound.

A decrease in activity in the presence relative to the absence of the test compound may be indicative that the compound is a DNPH1 inhibitor that is potentially useful in the treatment of HR cancer in combination with a PARP inhibitor.

Suitable methods for determining DNPH1 activity are well-known the art.

In other embodiments, a method of screening for a compound useful in the treatment of an HR cancer in combination with a PARP inhibitor or a 5-modified-2′-deoxypyrimidine nucleoside, may comprise; determining the expression of DNPH1 in a mammalian cell in the presence and absence of a test compound.

A decrease in expression of DNPH1 in the cell in the presence relative to the absence of the test compound may be indicative that the compound is a DNPH1 antagonist that is potentially useful in the treatment of HR cancer in combination with a PARP inhibitor.

Suitable methods for determining DNPH1 expression are well-known the art.

In some embodiments, a method of screening for a compound useful in ameliorating toxicity in an organ or tissue of an individual undergoing treatment with a combination of a PARP inhibitor and a 5-modified-2′-deoxypyrimidine nucleoside may comprise determining the activity of an isolated SMUG1 in the presence or absence of a test compound.

A decrease in SMUG1 activity in the presence relative to the absence of the test compound is indicative that the test compound is useful in ameliorating toxicity in an organ or tissue of an individual undergoing treatment with a combination of a PARP inhibitor and a 5-modified-2′-deoxypyrimidine nucleoside.

In other embodiments, a method of screening for a compound useful in ameliorating toxicity in individual undergoing treatment with a combination of a PARP inhibitor and a 5-modified-2′-deoxypyrimidine nucleoside may comprise determining the expression of SMUG1 in a mammalian cell in the presence and absence of a test compound.

A decrease in expression of SMUG1 in the cell in the presence relative to the absence of the test compound may be indicative that the compound is a SMUG1 antagonist that is potentially useful in ameliorating toxicity in an organ or tissue of an individual undergoing treatment with a combination of a PARP inhibitor and a 5-modified-2′-deoxpyrimidine nucleoside.

The precise format of any of the screening or assay methods of the present invention may be varied by those of skill in the art using routine skill and knowledge. The skilled person is well aware of the need to employ appropriate control experiments.

A test compound may be an isolated molecule or may be comprised in a sample, mixture or extract, for example, a biological sample. Compounds which may be screened using the methods described herein may be natural or synthetic chemical compounds used in drug screening programmes. Extracts of plants, microbes or other organisms, which contain several characterised or uncharacterised components may also be used.

Suitable test compounds include analogues, derivatives, variants and mimetics of a DNPH1 or SMUG1 substrate, for example compounds produced using rational drug design to provide test candidate compounds with particular molecular shape, size and charge characteristics suitable for modulating DNPH1 or SMUG1 activity.

Combinatorial library technology provides an efficient way of testing a potentially vast number of different compounds for ability to modulate DNPH1 activity. Such libraries and their use are known in the art, for all manner of natural products, small molecules and peptides, among others. The use of peptide libraries may be preferred in certain circumstances.

The amount of test compound which may be added to an assay of the invention will normally be determined by trial and error depending upon the type of compound used. Typically, from about 0.001 nM to 1 mM or more concentrations of putative inhibitor compound may be used, for example from 0.01 nM to 100 μM, e.g. 0.1 to 50 μM, such as about 10 μM. Even a compound which has a weak effect may be a useful lead compound for further investigation and development.

A test compound identified as modulating DNPH1 or SMUG1 expression or activity may be investigated further. For example, the selectivity of a compound for DNPH1 may be determined by screening against other isolated enzymes. Suitable methods for determining the effect of a compound on the activity of recombinant enzymes are well known in the art.

A test compound identified as a DNPH1 or SMUG1 antagonist may be isolated and/or purified or alternatively, it may be synthesised using conventional techniques of recombinant expression or chemical synthesis. Furthermore, it may be manufactured and/or used in preparation, i.e. manufacture or formulation, of a composition such as a medicament, pharmaceutical composition or drug. Methods described herein may thus comprise formulating the test compound in a pharmaceutical composition with a pharmaceutically acceptable excipient, vehicle or carrier for therapeutic application.

Following identification of a DNPH1 or SMUG1 antagonist that is potentially useful in the treatment of HR deficient cancer as described herein, a method may further comprise modifying the compound to optimise its pharmaceutical properties. Suitable methods of optimisation, for example by structural modelling, are well known in the art. Further optimisation or modification can then be carried out to arrive at one or more final compounds for in vivo or clinical testing.

Other aspects of the invention provide methods of determining the sensitivity or predicting the response of an HR deficient cancer in an individual to a combination of active agents. The combination of active agents may be selected from (i) a PARP inhibitor and an agent that reduces DNPH1 activity; (ii) a PARP inhibitor and a 5-modified-2′-deoxypyrimidine nucleoside, (iii) an agent that reduces DNPH1 activity and a 5-modified-2′-deoxypyrimidine nucleoside and (iv) a PARP inhibitor, an agent that reduces DNPH1 activity and a 5-modified-2′-deoxpyrimidine nucleoside.

In some embodiments, a method may comprise;
determining the expression of SMUG1 in a sample of HR deficient cancer cells obtained from the individual,
wherein a decrease in SMUG1 expression in the cancer cells relative to control cells may be indicative that the HR deficient cancer is insensitive or has reduced sensitivity to the combination of active agents and/or the individual is not responsive to the combination.

In other embodiments, a method may comprise;
determining the expression of DNPH1 in a sample of HR deficient cancer cells obtained from the individual,
wherein a decrease in DNPH1 expression in the cancer cells relative to control cells may be indicative that the HR deficient cancer is sensitive or has increased sensitivity to the combination of active agents relative to control cells and/or the individual is responsive to the combination.

Methods of determining SMUG1 and DNPH1 expression in samples of cells are well known in the art and include Northern blotting, RT-PCT, SAGE or RNA-seq.

In other embodiments, a method may comprise;
determining the intracellular concentration of hmdU in a sample of HR deficient cancer cells obtained from the individual,
wherein an increase in intracellular concentration of hmdU in the cancer cells relative to control cells may be indicative that the HR deficient cancer is sensitive or has increased sensitivity to the combination of active agents relative to control cells and/or the individual is responsive to the combination.

Suitable methods of determining the concentration of hmdU in samples of cells are well known in the art

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

### Experimental

### Cell Lines and Culturing

The human haploid chronic myeloid leukemia cell line eHAP (37) was purchased from Horizon Discovery. All eHAP cell lines used in this study were cultured as diploids. DLD1 WT and BRCA2−/− were from Horizon Discovery and SUM149 parental (BRCA1 C.2288delTp.N723FsX13), revertant (c.[2288delT, 2293del80]) (23), SUM149 53BP1−/− and SUM149 PARP1−/− were a gift from Christopher Lord (ICR, London). All cells were grown in humidified incubators at 37° C. and 10% CO2. eHAP cells were cultured in IMDM (Gibco) supplemented with 10% FBS (Sigma) and Penicillin-Streptomycin (Sigma). DLD1 cells were cultured in DMEM supplemented with 10% FBS and Penicillin-Streptomycin. SUM149 cells were cultured in Hams F-12 media (Gibco).

### Generation of KO Cell Lines

Isogenic cell lines were generated using CRISPR/Cas9 mutagenesis of eHAP cells, a haploid cell line derived from KBM-7 chronic myeloid leukemia (CML) cells, DLD1 WT and BRCA2 −/− and SUM149 parental (BRCA1 C.2288delTp.N723FsX13) and SUM149 revertant (c.[2288delT, 2293del80]) (Drean et al., 2017). To generate knock-out cell lines, cells were transfected with pX459V2-puro vector carrying the sgRNA targeting sequences using Fugene HD (eHAP) Lipofectamine 2000 (DLD1 and SUM149). After 24 hours, cells were selected with 0.4 μg/ml (eHAP) or 1 μg/ml (DLD1 and SUM149) puromycin for 2-4 days and then seeded as single cells for clonal selection. Clones were picked and knock-out was validated using sequencing and immunoblotting. The following sequences of sgRNA target sequences were used for CRISPR-Cas9: MUS81: 5′-TACTGGCCAGCTCGGCACTC-3′, DNPH1: 5′-TCATAGCCTACACCCAAGGA-3′ and SMUG1: 5′-CGAGTCACGTAGTTGCGATG-3′.

### Cloning and Lentiviral Expression of Proteins

MUS81, DNPH1 and SMUG1 cDNAs were cloned into the pCR8 gateway entry vector using the pCR™8/GW/TOPO™ TA Cloning Kit (Invitrogen). Phusion Site-Directed Mutagenesis Kit (Thermo Scientific) was used to generated nuclease dead MUS81D338A/D339A, and the active site mutants DNPH1E105A and SMUG1G87Y. cDNA from entry vectors were transferred to pLenti CMV Puro DEST vector (#17452, Addgene) using Gateway LR Clonase II kit (Invitrogen). For lentiviral production, 293FT cells (Invitrogen) were co-transfected with the individual pLenti CMV puro plasmids and packaging plasmids pLP1, pLP2 and pLP/VSVG (Invitrogen) using Lipofectamine 2000. Three days post transfection, the lentiviral-containing supernatant was collected and filtered. Cells were transduced and selected by puromycin followed by expansion of single clones. Protein expression was verified by western blotting.

### Genome-Wide CRISPR-Cas9 Screen

eHAP WT and MUS81−/− isogenic cells were transduced with a genome-wide lentiviral lentiCRISPRv2 sgRNA library (Doench et al 2016, PMID: 26780180) using multiplicity of infection (MOI) of ˜0.5 with a complexity of 400 cells/sgRNA. Transduced cells were selected with puromycin (0.4 mg/ml), expanded for 7 days, and sub-cultured in the presence or absence of an LD80 dose of olaparib (200 nM) or hmdC (1000 nM) for 10 days. Surviving cells were collected and genomic DNA was extracted using DNeasy Blood & Tissue Kit (Qiagen) supplemented with RNAse (Qiagen) according to the manufacturer's instructions. The CRISPR screens were carried out in biological triplicates. Libraries for Illumina sequencing were generated by two-step PCR amplification. In the first PCR, the sgRNA library was amplified and adaptors were added using nesting primers and the second PCR added barcodes, stagger regions, flow cell attachment and Illumina sequencing primers.

### Analysis of Genome Wide CRISPR-Cas9 Screen

After PCR amplification and sequencing, coverage-normalised read counts for the surviving population from the olaparib exposed cohort were compared to read counts from the vehicle population. Screens were analysed using the Mageck robust rank aggregation algorithm (Li et al., 2014 Genome Biol. 15, 554) to generate limit fold change and p-value for enrichment in the surviving population.

### Illumina Library Generation, Sequencing and Bioinformatic Analyses

Libraries were quantified using Qubit (Thermo Fisher) and TapeStation (Agilent) and were normalized and pooled for sequencing on a HiSeq 4000 system (Illumina) in a single ended read configuration, with a minimum length of 75 bp reads. Raw data was trimmed by obtaining 20 bp after the first occurrence of “CACCG” in the read sequence. Trimmed reads were then mapped with BWA version 0.5.9-r16 (38) to a database of guide sequences for the human CRISPR Brunello lentiviral pooled library with the parameters “-l 20 -k 2 -n 2”. sgRNA counts were obtained after filtering the mapped reads for those that had zero mismatches and mapped to the forward strand of the guide sequence. The MAGeCK ‘test’ command version 0.5.7 (39) was used to perform the sgRNA ranking analysis between the relevant conditions with parameters “--norm-method total --remove-zero both”. Coverage-normalized read counts for the surviving population from the olaparib or hmdC exposed cohorts were compared to read counts from the mock treated population. Screens were analyzed using the MAGeCK robust rank aggregation algorithm (39) to generate limit fold change (LFC) and corresponding statistical score (p-value) for each gene to identify both positively and negatively selected genes simultaneously (Table 51). Screen datasets were further subjected to bioinformatical analysis by STRING and Gene Ontology with an LFC cut-off of <−1.5 and statistical score <0.05.

### Cell Viability Assays

For analysis of cell viability following exposure to olaparib (Selleckchem), deoxyribonucleosides and DNPH1i (N6-benzyladenosine, Carbosynth), cells were seeded in 96-well plates at a density of 100-500 cells per well. Twenty-four hours post-seeding, drug treatments were initiated, and cells were continuously exposed until termination of the experiment. Cell viability was measured using CellTiter-Glo (Promega). The surviving fractions were measured, and drug sensitivity curves were plotted using GraphPad Prism. The following nucleosides were purchased from commercial sources: dU (2′-deoxyuridine), hdU (5-hydroxy-2′-deoxyuridine), hmdU (5-hydroxymethyl-2′-deoxyuridine), mdC (5-methyl-2′-deoxycytidine), hmdC (5-hydroxymethyl-2′-deoxycytidine), cadC (5-carboxy-2-deoxycytidine), fodC (5-formyl-2-deoxycytidine), dl (2′-deoxyinosine), dX (2′-deoxyxanthosine), 8odG (8-oxo-2′-deoxyguanosine), hmU (5 hydroxymethyluridine), hmC (5-hydroxymethyl-cytidine). fodU (5-formyl-2′-deoxyuridine) was synthesized as previously described (Guo et al Org. Biomol. Chem. 11, 1610-1613 (2013).

### Analysis of Nucleotide Composition in Genomic DNA

Cells (5×10⁷) were collected and DNA extracted using Gentra Puregene Cell Kit (Qiagen) supplemented with RNAse (Qiagen) and 100 mM tetrahydrouridine (Merck). DNA was digested to mononucleosides by modification of a published protocol (41). Briefly, reactions were carried out in 100 ml buffer containing 150 mg DNA, 50 mM Tris pH 9.0, 20 mM NaCl, 5 mM MgCl2, 1 kU Benzonase, 1 U phosphodiesterase I and 10 U FastAP alkaline phosphatase for 48 hrs at 37° C. The samples were then precipitated with chloroform and labelled standards of deoxyadenosine (dA), deoxycytidine (dC), deoxyguanosine (dG) and deoxythymidine (dT) were spiked for normalization of the signal response from modified deoxynucleosides. Polar phases were analyzed by LC-MS/MS using a using a reverse phase column (100×2.1 mm, 3 μm, Fortis C18, Fortis Technologies) on a Dionex UltiMate LC system coupled to a TSQ Quantiva™ Triple Quadrupole (Thermo Scientific) mass spectrometer with a HESI 11 (heated electrospray ionization) source. The mobile phases consisted of water containing 0.1% acetic acid (mobile phase A) and acetonitrile (mobile phase B). A gradient of 12 min 0-12% B and 1 min 12-60% B was used for the separation of all the modified nucleosides. MS parameters were as follows: spray voltage 3.5 kV and 2.5 kV for positive and negative mode, respectively; temperature of capillary and vaporizer gas was 375° C. and 275° C.; sheath and auxiliary gases were 35 and 10 arbitrary units, respectively; the CID gas was set at 1.5 mTorr. Analysis was performed in selected reaction monitoring mode. Collision energies were manually optimized using commercial standards and dwell times were set to 40 ms for each transition. Nucleoside isotopes were purchased from Goss Scientific: 2′-deoxyguanosine (15N5), 2′-deoxyadenosine (15N5), 2′-deoxycytidine (15N3) and thymidine (15N2).

### DNPH1 Purification

HIS6DNPH1 and HIS6DNPH1E105Q, cloned in pET28a were transformed into BL21 cells. Transformed cells were inoculated into 2 L of TB media supplemented with 50 μg/ml kanamycin and incubated 37° C. until an OD600=1.0. IPTG (0.5 mM) was added and incubation continued for 4 hours. Cells were harvested by centrifugation and resuspended in lysis buffer (50 mM HEPES pH 7.5, 300 mM NaCl, 5% glycerol, 0.5 mM TCEP) supplemented with protease inhibitors (Complete EDTA-free tablets, Roche). Cells were lysed by passage through an Emulsiflex C5 (Avestin) at 150,000 psi, and the chromatin sheared by sonication at 50% maximum amplitude for 2×30 sec. Soluble proteins were isolated by ultracentrifugation (Beckman Type JLA25.50 rotor) at 60,000×g for 60 min (4° C.). Soluble extract was supplemented with 30 mM imidazole and incubated with 2 ml Ni-NTA beads (GE Healthcare) for 1 hour (4° C.). The beads were washed (50 mM HEPES pH 7.5, 300 mM NaCl, 30 mM imidazole, 5% glycerol, 0.5 mM TCEP) and protein eluted (50 mM HEPES pH7.5, 300 mM NaCl, 200 mM imidazole, 5% glycerol, 0.5 mM TCEP). The eluates were pooled, diluted dropwise to 100 mM NaCl (50 mM HEPES pH 7.5, 5% glycerol, 0.5 mM DTT) and DNPH1 further purified using a 5 ml HiTRAP QHP column (GE Healthcare), followed by a Superdex 200 Increase 10/300 column (GE Healthcare). Peak fractions were separated by SDS-PAGE and visualized with Instant Blue (Gentaur). Peak fractions were frozen in aliquots and stored at −80° C.

### DNPH1 In Vitro Activity Assays

DNPH1 (4 μM) was incubated with nucleoside monophosphates (1 mM) in 20 mM sodium phosphate, pH 7.0, 150 mM NaCl, 2 mM MgCl2. After 45 min at room temperature the reaction was quenched by addition of 0.7% perchloric acid followed by immediate neutralization with addition of sodium acetate to 200 mM. Samples were diluted 1:10 into RP-Buffer (100 mM K2HPO4/KH2PO4, pH 6.5, 10 mM tetrabutylammonium bromide, 7% acetonitrile) and the nucleotides and reaction products separated from precipitated protein by filtration through a 0.22 μm centrifugal filter (Durapore-PVDF, Millipore). Samples of each reaction (5 nmol) were applied to a Zorbax SB-C18 column (4.6×250 mm, 3.5 μm, 80A pore size, Agilent Technologies), maintained at 30° C. and mounted on a Jasco HPLC system controlled by Chromnav software (v1.19, Jasco). Nucleoside monophosphates and reaction products were separated under isocratic flow by application of RP-Buffer at 1 mL/min. Absorbance data from the column eluent was continuously monitored between 200-400 nm (2 nm interval) using an MD-2010 photodiode array detector (JASCO). Nucleoside monophosphate and nucleobase peaks were identified by comparison with the retention times and UV/Vis spectra of standards. Peak integration of the absorbance data recorded at 260 nm was used to quantify the amount of substrate and product. For the determination of the rate of hmdUMP hydrolysis by DNPH1, samples were withdrawn from a reaction containing DNPH1 (2 μM) and quenched at intervals up to 20 minutes. The rate was determined by linear regression of a plot of product against reaction time.

### Immunoblotting

Cells were lysed on ice in cold EBC-buffer (150 mM NaCl, 50 mM Tris-HCl pH 7.4, 1 mM EDTA, 0.5% NP-40/Igepal) supplemented with cOmplete Protease Inhibitor Cocktail (Roche), PhosSTOP (Roche) and 1 mM dithiotreitol. The lysates were sonicated using a Bioruptor Plus with 10 cycles of 30′ OFF/30′ ON, high (Diagenode). Proteins were separated by SDS-PAGE and transferred onto a PROTRAN 0.2 μM nitrocellulose membrane (Life Technologies) using wet transfer. Blocking and blotting with primary antibodies were performed in PBS-T supplemented with 5% skimmed milk powder. Proteins were visualized on films using secondary HRP-conjugated antibodies (Dako) and chemiluminescent detection using ECL (GE Healthcare).

### Pulsed-Field Gel Electrophoresis (PFGE)

DNA break formation was analyzed by PFGE as described (42). For each sample, 1×106 cells were collected after treatment and cast into agarose plugs (low gelling temperature, Sigma-Aldrich). Plugs were deproteinized by incubation in proteinase K buffer (0.5 M EDTA, 1% N-laurylsarcosyl, 1 mg/ml proteinase K) at 50° C. for 24 h. Plugs were washed three times in TE buffer (10 mM Tris-HCL pH 8.0, 50 mM EDTA) and loaded onto a 1% agarose gel (pulsed-field grade, Bio-Rad) and separated by PFGE for 20 h at 120° angle, 60-240 s switch time, 4 V/cm at 14° C. (CHEF DR III, Bio-Rad). The gel was stained with ethidium bromide and visualized using Quantity One software (Bio-Rad).

### Antibodies

The following antibodies were used: PARP1 (#9542, Cell Signaling), SMUG1 (ab192240, Abcam), DCTD (ab183607, Abcam), BRCA1 (#07-434, Millipore), KAP1 (ab10483, Abcam), pKAP1 S824 (ab70369, Abcam), pCHK1 S317 (#2344, Cell Signaling), GEN1 (SCY6, Francis Crick Institute), H2A (#2578, Cell Signaling), Actin (ab8226, Abcam), RAD51 (SWE47, Francis Crick Institute), BRCA2 (OP95, Calbiochem), γ H2AX (Millipore, JBW301), DNPH1 (sc-365682, Santa Cruz), MUS81 (MTA30, Francis Crick Institute), 53BP1 (612522, BD Biosciences), RPA2 (ab2175, Abcam), HA-tag (3F10, Roche).

### Immunofluorescence

DLD1 isogenic cell lines were plated at 5000 cells per well into 96 well plates. After 24 h, the cells were exposed to olaparib, hmdU or a combination of olaparib and hmdU. 48 hours post drug exposure, cells were washed 3× in PBS, fixed in 4% PFA for 10 minutes and washed 3× in PBS. Cells were permeabilized using 0.5% Triton-X in PBS for 10 minutes, and incubated with IFF buffer (1% FBS, 2% BSA in PBS) for 1 h. They were then incubated with primary antibodies targeting gH2AX and RAD51 in IFF buffer overnight at 4° C. Primary antibodies were detected with Alexa Fluor-555 and Alexa Fluor-488 conjugated secondary antibodies for 1 h at room temperature. DAPI staining was used to detect nuclei. Nuclear foci were visualized using high content screening system (Opera Phoenix, PerkinElmer), using a 40× lens. Non-biased focus analysis was performed using the Harmony 4.9 software, which allows for threshold-based quantification of nuclear foci. At least 1000 cells were counted per condition, per biological replica. The mean number of foci per cell was used in the final analysis for three biological replicates per cell lines per condition.

### Gene Ontology and Interaction Network Analyses

For Gene Ontology analysis, PANTHER (43, 44) (http://pantherdb.org) was used for biological pathway enrichment of screen hits with an LFC of <−1.5 and a p<0.05. PANTHER pathway analysis was performed against the Reactome version 65 Released 2019-03-12 database. Analysis was performed using a Fisher's Exact test with a Bonferroni correction for multiple testing (Bonferroni-corrected for p<0.05). Mapping of the hits on protein interaction networks was done through STRING (https://string-db.org) with a minimum required interaction score of 0.4.

### PARP Trapping Assay

The chromatin fractionation assay for PARP trapping was performed as previously described (Murai et al, 2012, PMID: 23118055). Briefly, 500.000 cells were seeded onto six-well plates, and treated with 350 nM hmdU or 0.01% MMS for 24 hours at which point cells were either left untreated or treated with 10 μM olaparib for 4 h. Cells were fractionated with the Subcellular Protein Fractionation kit for Cultured Cells (ThermoFisher #78840) according to the manufacturer's instructions. Soluble and chromatin fractions were analysed by immunoblotting for PARP1 (Cell Signaling #9542, rabbit) and yH2AX (Millipore, JBW301) antibodies. Incubation with primary antibody was followed by incubation with a horseradish peroxidase-conjugated secondary antibody and chemiluminescent detection of proteins (Amersham Pharmacia, Cardiff, UK).

### Statistical Analysis

Data from the genome wide CRISPR screen was processed as detailed in the text and elsewhere (39). In total, we conducted three biological replicas of the screen and used MAGeCK to calculate log fold change and p values for each sgRNA from the replica data. Data from comparative groups in the in vitro drug sensitivity assays was compared using ANOVA (either two way or two-way repeated measures, as appropriate) in the Graphpad Prism software package or Student's t-test where appropriate. False discovery rate method of two-stage linear step-up procedure of Benjamini, Krieger and Yekutieli (45) was used as post-test with the desired FDR (Q)=1%. At least three biological replicates were performed from each experiment. Error bars represent standard error of the mean (s.e.m.).

### Results

### Genome-Wide CRISPR-Cas9 Screen in HR Deficient Cells Identifies DNPH1 as a PARP Inhibitor Sensitizer

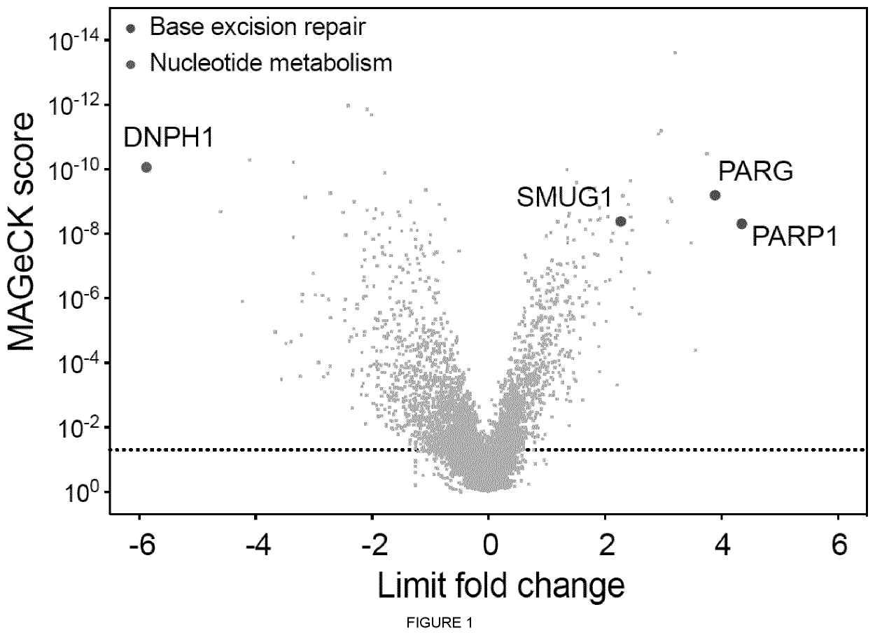

MUS81 k/o cells were transduced with the lentiviral-based Brunello genome-wide gRNA library (J. G. Doench et al Nat. Biotechnol. 34, 184-191 (2016)), followed by treatment with olaparib for 10 days. The LD80 dose used allowed us to identify both dropout (sensitizing) and enriched (resistance causing) gRNAs. Bioinformatic analysis of the gRNA reads using the MAGeCK algorithm identified several novel, as well as previously reported, factors determining PARPi efficacy (FIG. 1).

PARPi resistance was observed with gRNAs that targeted PARP1 and the de-PARylation factor PARG, which promote resistance by rescuing PARP trapping (J. Murai et al., Cancer Res. 72, 5588-5599 (2012); S. J. Pettitt et al. Nat. Commun. 9, 1849 (2018)) or restoring PARP activity (E. Gogola et al., S. Cancer Cell 33, 1078-1093 (2018)). Sensitizing gRNAs included several BER factors (POLB, LIG3, RFC1, HPF1 and ALC1), indicating that defective BER is synthetic lethal with PARPi, likely through increased PARP trapping on BER intermediates. Furthermore, Gene Ontology and STRING protein interaction analyses revealed an enrichment of DNA repair enzymes involved in BER and the Fanconi anemia pathway, as observed previously (K. E. Mengwasser et al. Mol. Cell 73, 885-899 (2019)). Inactivation of factors involved in in NADH metabolism and mitochondrial homeostasis also sensitized cells to PARPi, which could potentially lead to increased reactive oxygen species (ROS) as a result of mitochondrial dysfunction (D. B. Zorov, et al. Physiol. Revs. 94, 909-950 (2014)), induced DNA damage and PARP trapping (X. Luo, et al. Genes dev. 26, 417-432 (2012).

The highest ranking gene from the screen was the putative nucleotide sanitizer DNPH1/RCL (2′-deoxynucleoside 5′-monophosphate N-glycosidase), a c-Myc target that is overexpressed in various tumors (S. Shin, et al J. Cell. Biochem. 105, 866-874 (2008); B. C. Lewis et al., Canc. Res. 60, 6178-6183 (2000)) (FIG. 1). Disruption of a second nucleotide sanitizer ITPA (inosine triphosphatase) (S. Lin et al. J. Biol. Chem. 276, 18695-18701 (2001)) also sensitized the MUS81 k/o's to PARPi. DNPH1 and ITPA were validated as bona fide hits using individual CRISPR-generated knock-out cell lines.

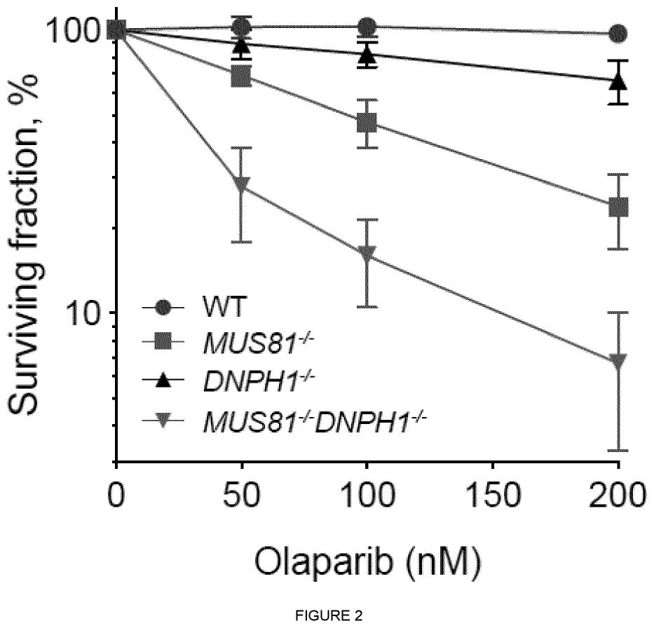

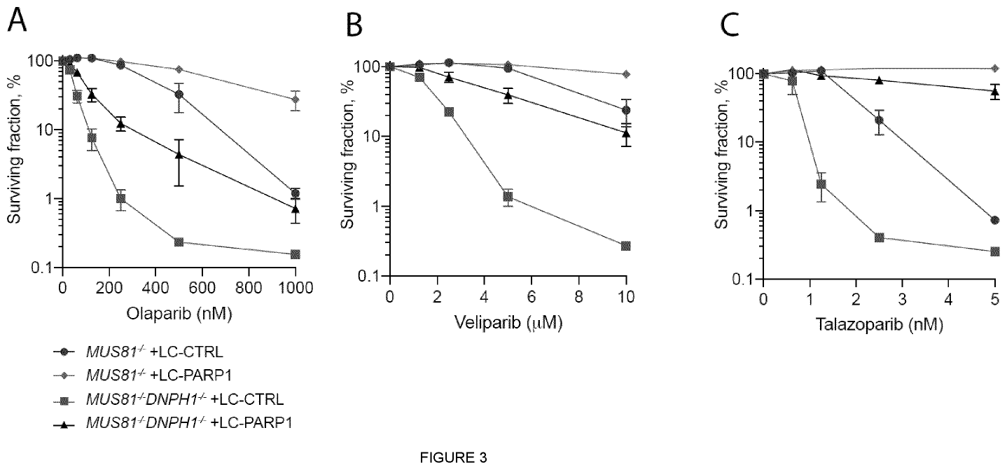

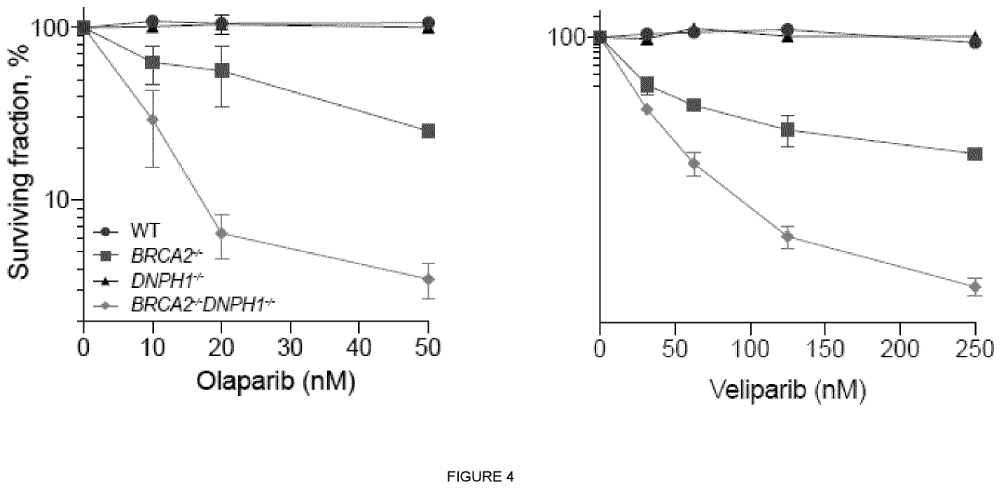

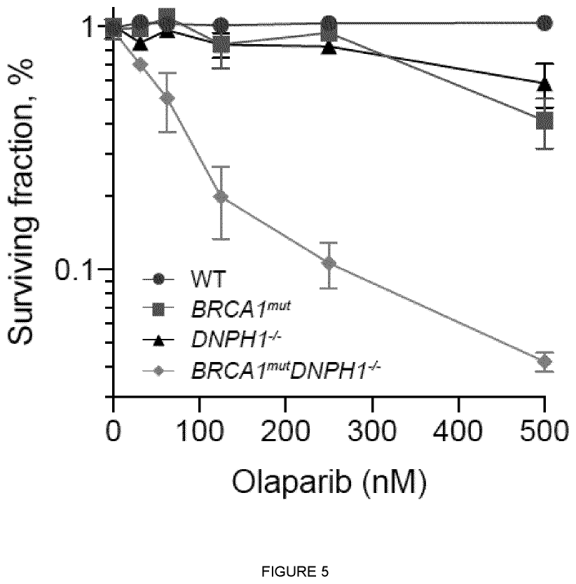

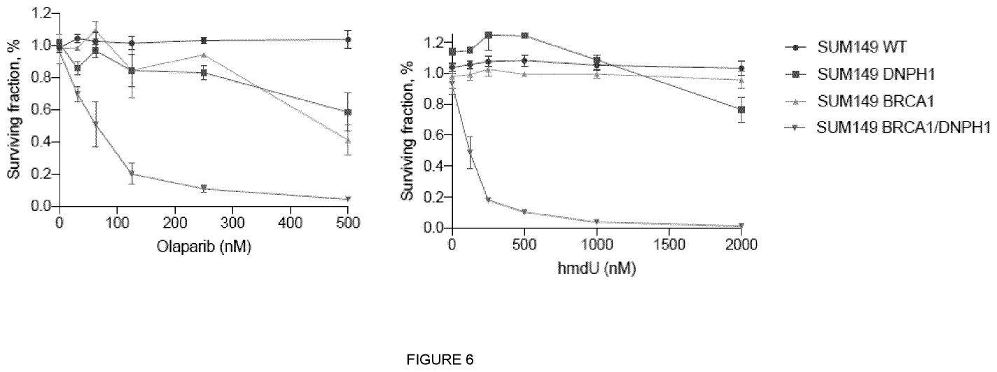

Disruption of either gene sensitized HR-deficient MUS81 k/o cells to treatment with olaparib (FIG. 2), indicating that their target nucleotides are sources of endogenous DNA lesions underlying PARPi cytotoxicity. We also found that loss of DNPH1 sensitised MUS81 KO cells to the other PARP inhibitors veliparib and talazoparib (FIG. 3). The marked sensitivity conferred by the DNPH1 k/o cells, combined with its uncharacterized biological function, led us to focus on the role of this gene in potentiating the effect of PARPi on HR-deficient cells. Importantly, we found that the DLD1 BRCA2-defective cell line and the SUM149 BRCA1-defective cell line, which provide more clinically relevant genetic backgrounds for HR-deficiency than MUS81 k/os, were both sensitized to PARPi by disruption of DNPH1 (FIGS. 4 to 6).

DNPH1 Targets hmdUMP to Limit Genomic Incorporation

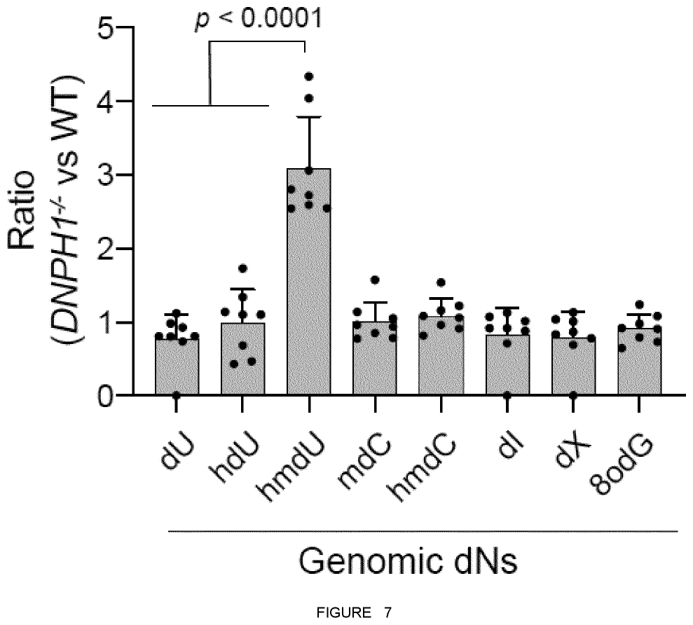

DNPH1 hydrolyses deoxyribonucleoside monophosphates (dNMPs) in vitro (Ghiorghi et al J. Biol. Chem. 282, 8150-8156 (2007)) but its biological nucleotide target(s) and function(s) remain unknown. We therefore carried out metabolomic analysis of the nucleoside composition of genomic DNA in wild-type and DNPH1 k/o cells and found a 3-4 fold increase in the level of genomic hmdU in the k/o cells (FIG. 7) but no significant change in other nucleosides. WT or DNPH1 k/o cells were therefore treated with hmdU to determine whether DNPH1 limits the incorporation of exogenous hmdU into DNA. We observed a 3-fold increase in genomic hmdU in the DNPH1 k/o cells. These results show that DNPH1 acts upon hmdUMP to preclude its incorporation into DNA.

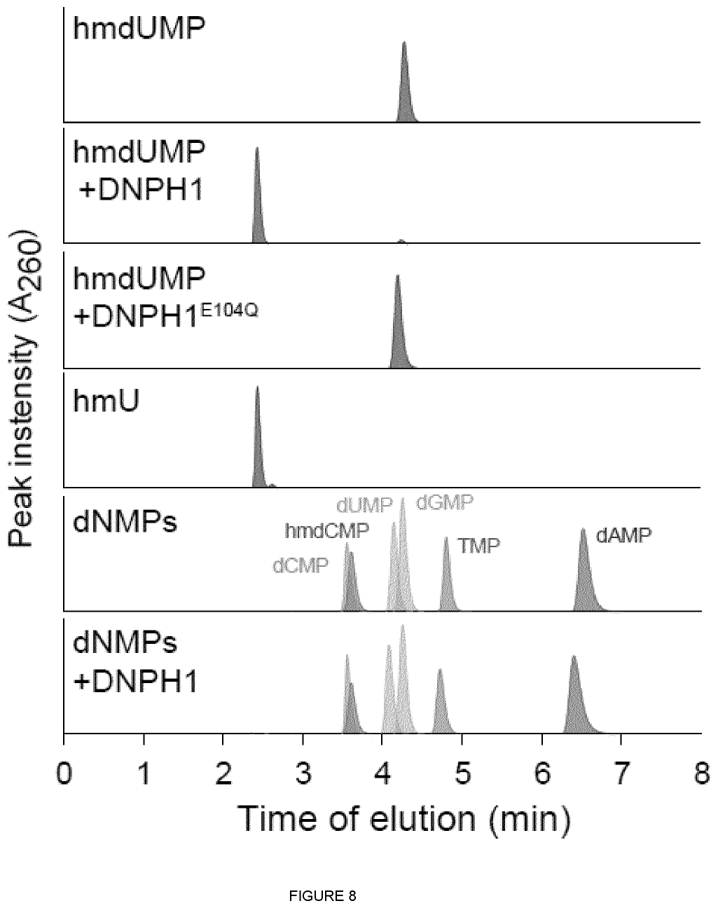

To determine whether DNPH1 can directly hydrolyze hmdUMP, we purified recombinant human DNPH1 and the active site mutant DNPH1E105Q. DNPH1, but not DNPH1E105Q, efficiently hydrolyzed hmdUMP to yield the hmU nucleobase with a reaction rate of 14.6+/−0.4 μM/min (FIG. 8). Little or no activity was observed towards any of the canonical dNMPs, dUMP or UMP (FIG. 8). These results support our in vivo data and confirm that hmdUMP is the direct biological target of DNPH1

hmdU is Synthetic Lethal with PARP Inhibition in HR Deficient Cells

DNPH1 is a putative nucleotide sanitizer that degrade aberrant nucleotides to prevent their incorporation into genomic DNA. As DNPH1 deficient cells are hypersensitive to PARP inhibition, we speculated that the synthetic lethality might be caused by PARP acting on a mis-incorporated aberrant nucleotide.

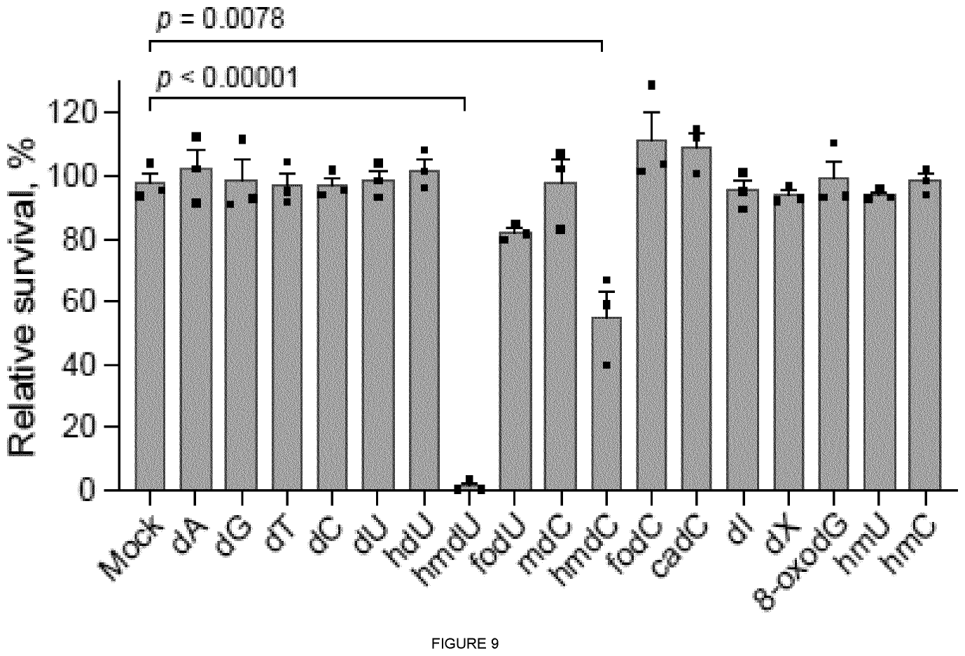

To further explore the biological function of DNPH1, we exposed MUS81 k/o or MUS81/DNPH1 k/o cells to a panel of deoxyribonucleosides carrying nucleobases modified by methylation, hydroxylation, deamination and oxidation, and measured cell viability. Strikingly, the HR-deficient MUS81/DNPH1 k/o's were hypersensitive to treatment with hdmU and to a lesser extent fodU and hmdC (FIG. 9), but not their ribonucleoside counterparts hmU or hmC, showing that the cytotoxic effect requires DNA incorporation. Complementation with DNPH1, but not a catalytic-dead mutant of DNPH1 (Ghiorghi et al J. Biol. Chem. 282, 8150-8156 (2007)), rescued this hmdU-induced cytotoxicity.

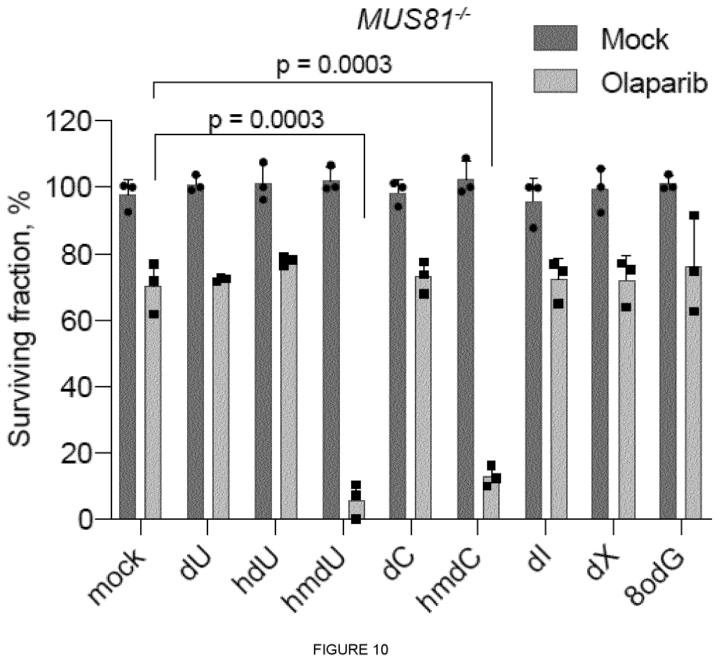

To determine whether the observed increase of genomic hmdU in DNPH1-deficient cells was responsible for the sensitization of MUS81 and BRCA2 k/o cells to PARPi, we exposed HR deficient MUS81−/− cells to the panel of various nucleosides (as nucleotides are not cell permeable) in the absence of presence of the PARP inhibitor olaparib. Treatment with either nucleosides or olaparib alone only had limited effect on viability, however the combined treatment with olaparib and either hmdU or its precursor hmdC exhibited a strong synthetic lethality (FIG. 10), showing that hmdU potentiates PARPi treatment.

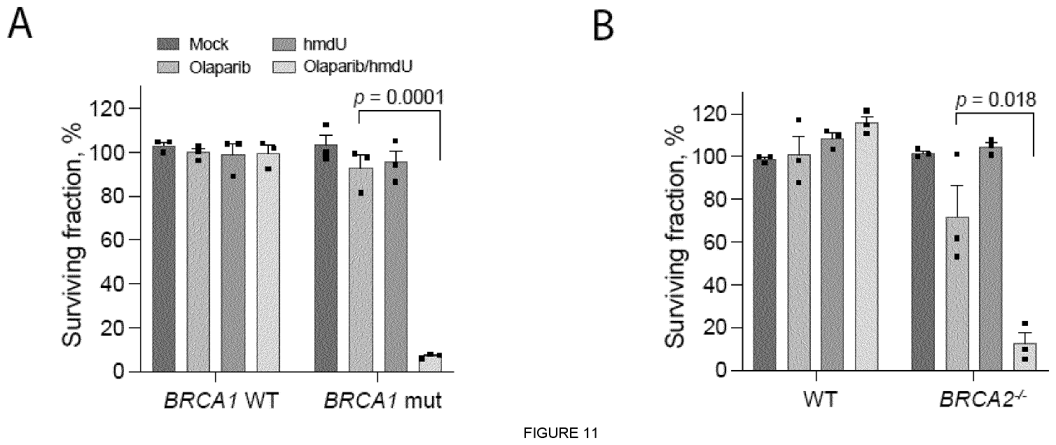

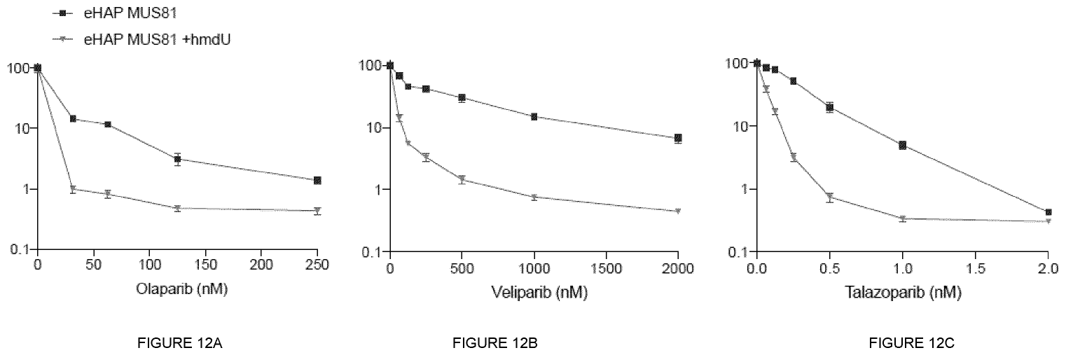

To verify if the observed synthetic lethality was indeed related to HR deficiency, we carried out the experiment with hmdU and olaparib in both the patient derived SUM149 BRCA1 mutant cell line as well as the DLD1 BRCA2−/− cell line. Whereas treatment with either hmdU or olaparib alone only had limited effect on viability, the combined treatment with olaparib and hmdU induced synthetic lethality in both HR deficient cell lines (FIG. 11). Similarly, whereas treatment of MUS81−/− cells with olaparib, veliparib or talazoparib alone only had limited effect on viability, the combined treatment with olaparib and hmdU, veliparib and hmdU, or talazoparib and hmdU induced synthetic lethality in the MUS81−/− cells (FIG. 12).

In contrast, isogenic revertant cells in which the reading frame of BRCA1 had been reverted to wild-type (Drean et al Mol. Cancer Ther. 16, 2022-2034 (2017)) were insensitive to hmdU/PARPi treatment (FIG. 11A). These results show that synthetic lethality can be induced in a variety of HR-deficient backgrounds and that DNPH1 and its substrate hmdU have potential for cancer therapy.

The Cellular Origins of hmdU

Nucleotide salvage pathways are generally employed as an energy efficient way to recycle deoxyribonucleosides that arise from the breakdown of DNA. To limit the reincorporation of nucleotides carrying epigenetic marks such as hmdC(MP), they are thought to undergo deamination to their uridine counterpart hmdU by cytidine deaminase (CDA) (Zauri et al Nature 524, 114-118 (2015).

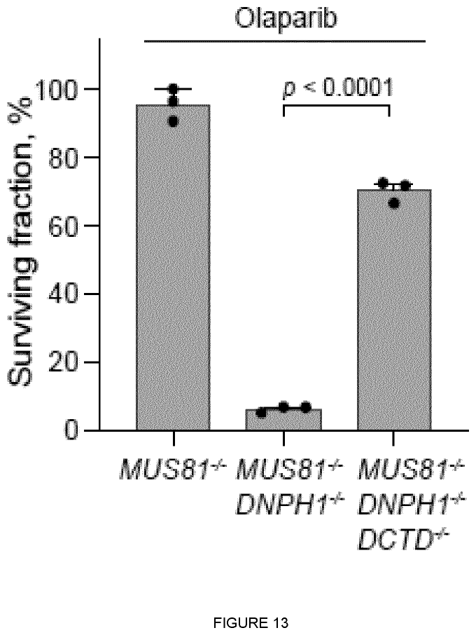

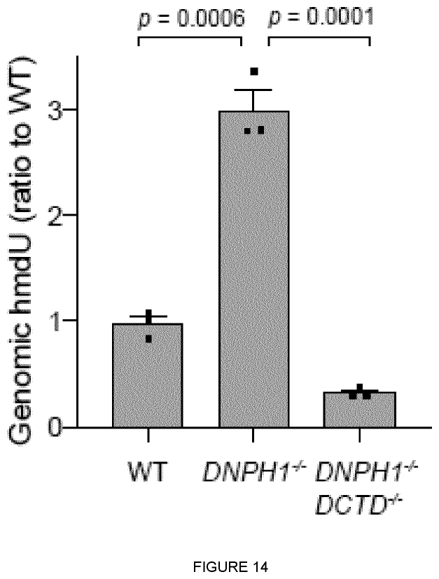

To determine whether hmdU arises from hmdC, and to identify factors involved in this pathway, we carried out a CRISPR screen in MUS81 k/o cells exposed to hmdC. Bioinformatic analysis revealed that the loss of factors involved in nucleoside activation by phosphorylation such as deoxycytidine kinase (DCK) and thymidylate kinase (DTYMK) rendered cells resistant to hmdC treatment. In contrast, gRNAs targeting DNPH1 sensitized cells to hmdC, again highlighting the critical role of DNPH1 in the survival of HR-deficient cells by eliminating hmdU. Remarkably, loss of the gene encoding the dCMP deaminase DCTD rendered cells completely resistant to hmdC, indicating that DCTD deaminates hmdC monophosphate (hmdCMP) to produce hmdUMP in the nucleotide pool. Loss of DCTD also conferred resistance to olaparib (FIG. 1), presumably as a consequence of decreased genomic hmdU. To test this possibility, we generated DCTD k/o cell lines and found that they were completely resistant to hmdC, but not hmdU treatment. Interestingly, the increased sensitization of MUS81 k/o cells to olaparib upon DNPH1 ablation was rescued by co-disruption of DCTD (FIG. 13), showing that cytotoxicity results from DCTD-mediated formation of hmdU. Consistent with this, the increase in genomic hmdU observed in DNPH1 k/o cells was rescued by disruption of DCTD (FIG. 14). These results support the hypothesis that hmdUMP, produced by DCTD-dependent deamination of hmdCMP, is the biological target of DNPH1. Loss of CDA did not confer resistance to either hmdC or olaparib, indicating that DCTD is the primary deaminase responsible for hmdUMP production from salvaged hmdC.

hmdU is Synthetic Lethal with Loss of DNPH1 Expression in HR Deficient Cells

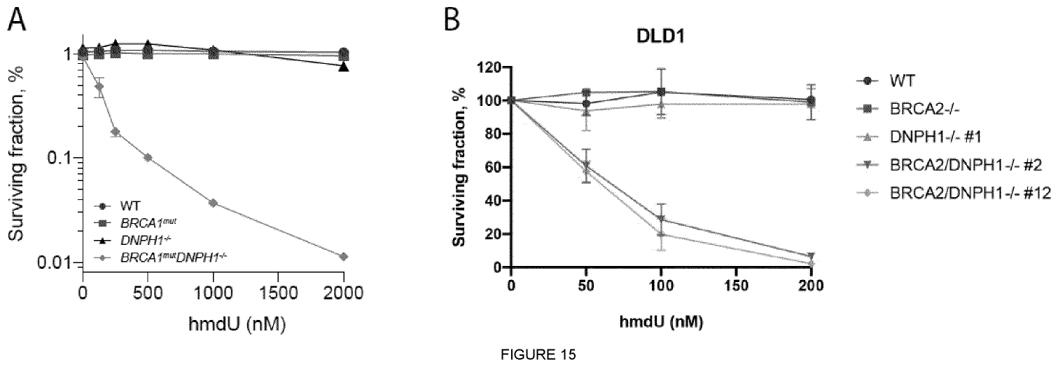

As loss of DNPH1 sensitizes HR deficient cells to PARP inhibition and the fact hmdU is synthetic lethal with PARP inhibition, we were interested in how DNPH1 deficient cells would react to hmdU treatment. To this end, we generated isogenic DNPH1 knock-out cell lines in either DLD1 WT or BRCA2−/− background (FIG. 15A). Using cell viability assays, we found that whereas WT, BRCA2 and DNPH1 single knock-out cell lines were resistant to the highest dose tested, two independent BRCA2/DNPH1 double knock-out cell lines were hypersensitive to treatment with hmdU, killing over 95% of the cells even in the absence of a PARP inhibitor (FIG. 15B).

hmdU is Synthetic Lethal with Chemical Inhibition of DNPH1 in HR Deficient Cells

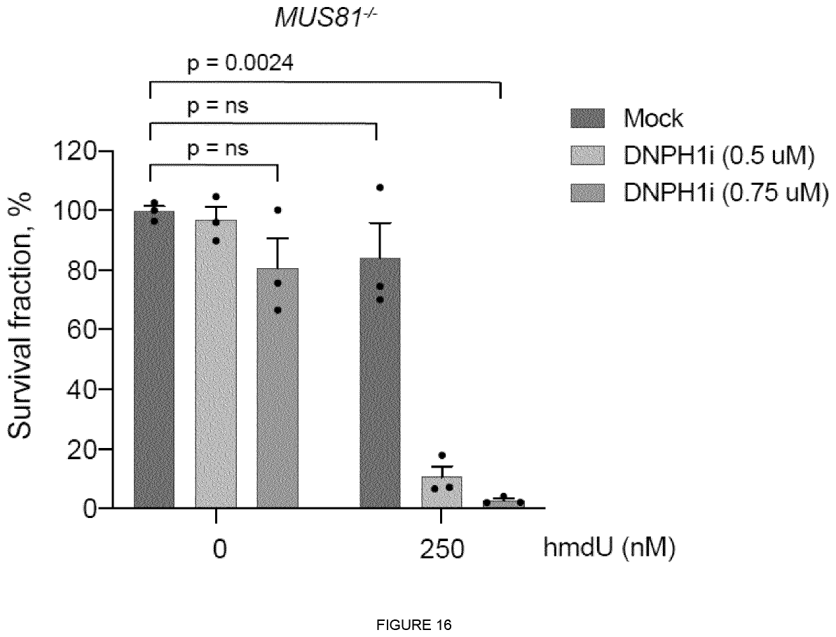

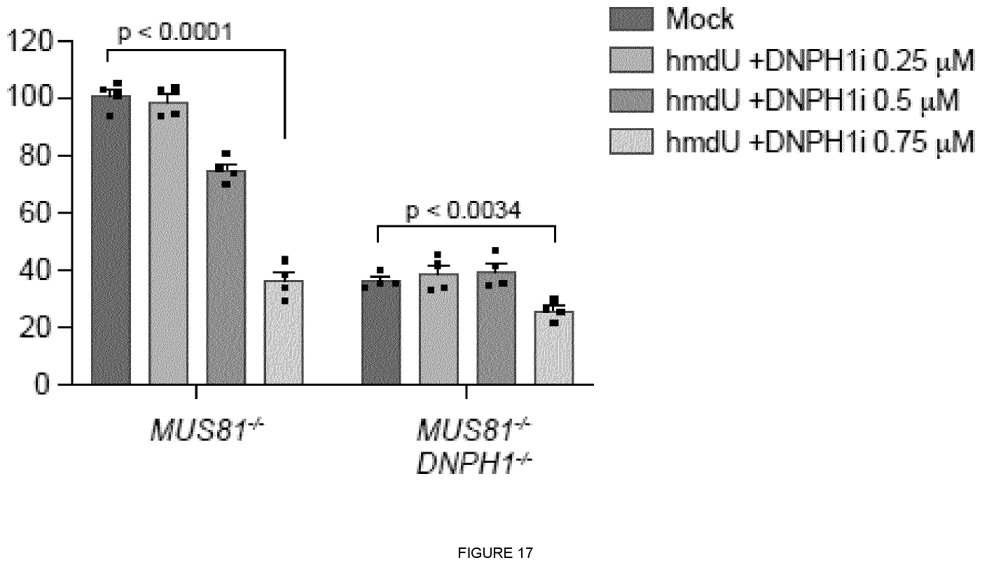

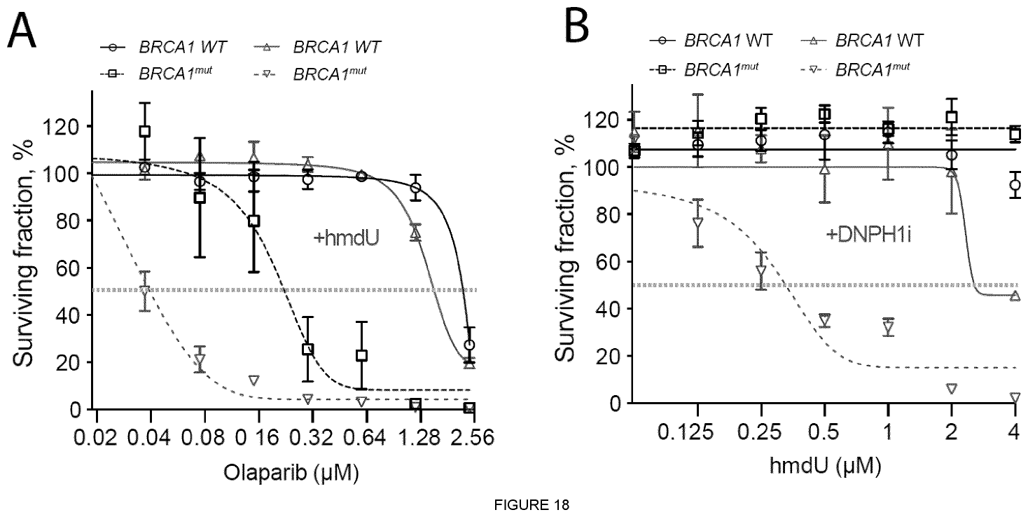

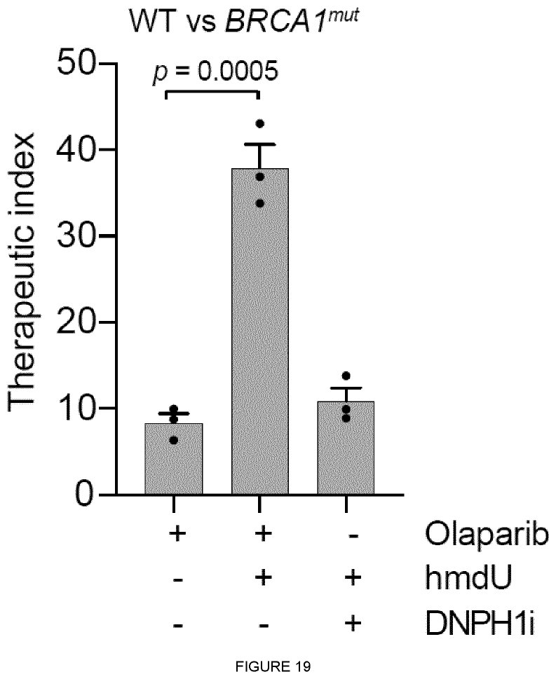

Since hmdU induces synthetic lethality in HR-deficient cells lacking DNPH1 (FIG. 15), we next determined whether it might be possible to induce synthetic lethality in BRCA deficient cells in a PARPi-independent manner. We used an existing competitive inhibitor of DNPH1, N6-benzyladenosine (DNPH1i; Amiable et al 2013). Treatment with either hmdU or N6AB alone in MUS81−/− cells had no effect on viability, however the combined treatment with hmdU and N6AB induced a strong synthetic lethality, killing over 95% of the cells (FIG. 16 Control experiments, in which wild-type cells showed a DNPH1i dose-dependent decrease in viability whereas DNPH1 k/o cells exhibited only a marginal effect, confirmed the specificity of DNPH1 i (FIG. 17). We further validated the synthetic lethality of DNPH1 inhibition in the patient derived SUM149 BRCA1 mutant cell line. Addition of hmdU greatly sensitized BRCA1-deficient cells to olaparib treatment, as compared to olaparib alone, through a 4-fold expansion of the therapeutic window (FIGS. 18A and 19). Furthermore, we found that DNPH1 inhibition induced synthetic lethality with hmdU to the same extent as Olaparib alone (FIGS. 18B and 19). The isogenic WT cells were unaffected by the treatment.

### Killing of PARPi-Resistant BRCA1 Deficient Cells

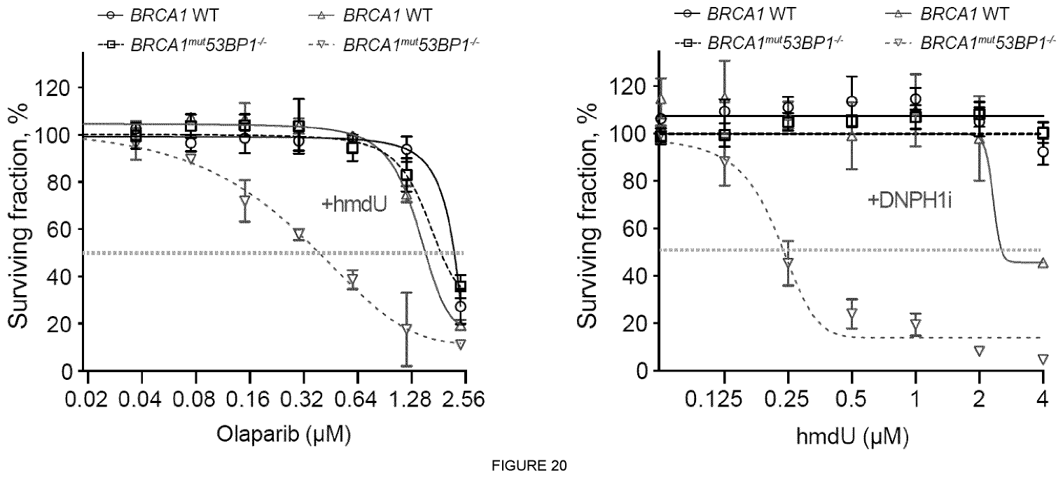

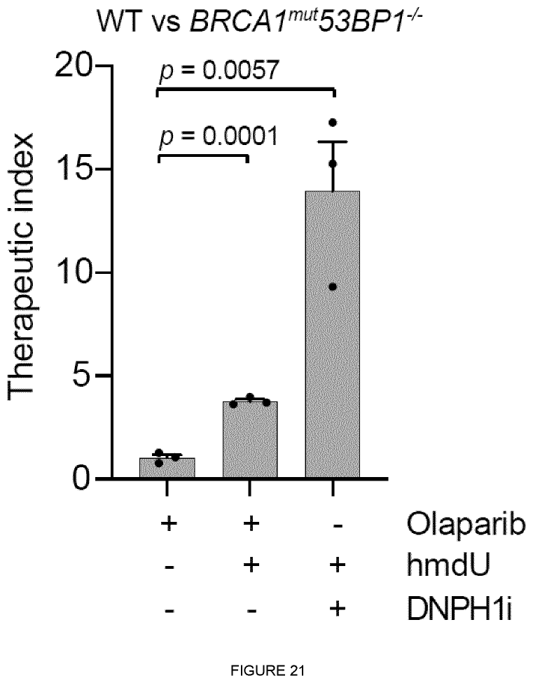

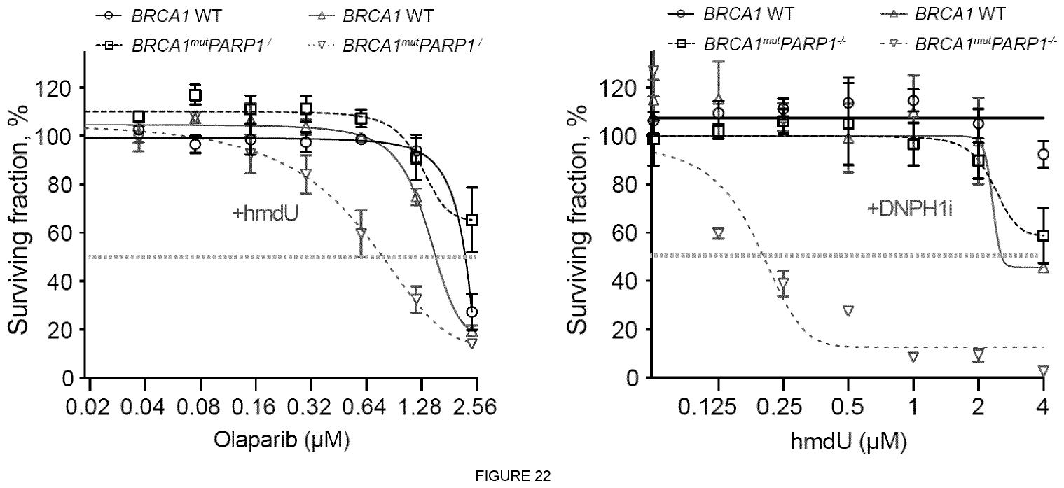

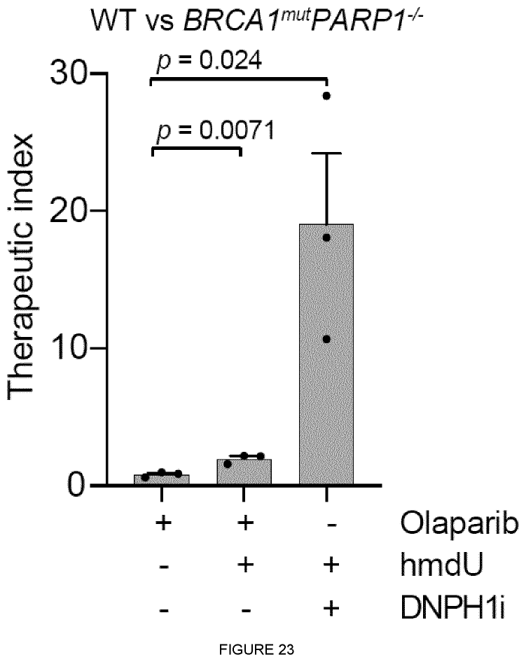

PARPi resistance arises by reversion of the BRCA genes to wild-type (Noordermeer et al Trends Cell Biol. 29, 820-834 (2019)), loss or mutation of PARP (Murai et al Cancer Res. 72, 5588-5599 (2012)), Pettitt et al Nat. Commun. 9, 1849 (2018)) or PARG (Gogola et al Cancer Cell 33, 1078-1093 (2018)), or in the case of BRCA1-deficient cells, by restoration of HR-proficiency through inactivation of the 53BP1-SHIELDIN pathway (Noordermeer et al Trends Cell Biol. 29, 820-834 (2019), Jaspers et al Cancer Discov. 3, 68-81 (2013)). To determine whether the potentiating effect of hmdU on PARPi in the killing of BRCA1-deficient cells could resensitize PARPi-resistant cells, we compared the effect of hmdU/olaparib in SUM149 cells carrying either BRCA1 mutant (parental) or wild-type (revertant), with or without knock-out alleles of PARP1 or 53BP1. We found that hmdU treatment was able to resensitized PARPi-resistant BRCA1mut/53BP1 cells (FIG. 20) and BRCA1mut/PARP1 (FIG. 22) to olaparib treatment by a 3-fold expansion of the therapeutic window (FIGS. 21 and 23).

Since hmdU induces synthetic lethality in HR-deficient cells lacking DNPH1 (FIG. 10), we next determined whether it might be possible to induce synthetic lethality in PARPi-resistant BRCA deficient cells in a PARPi-independent manner. Remarkably, combined treatment with hmdU and DNPH1 i efficiently killed PARPi-resistant BRCA1 cell lines lacking either PARP1 (FIG. 23) or 53BP1 (FIG. 21). Indeed, the therapeutic windows increased ˜10-fold and ˜20-fold, respectively, as compared to olaparib alone.

Collectively, these findings could potentially open up for new ways of killing HR deficient cancer cells independently of a PARP inhibitor, in particular those resistant to PARPi.

Loss of SMUG1 Promotes Resistance to hmdU/PARPi Treatment

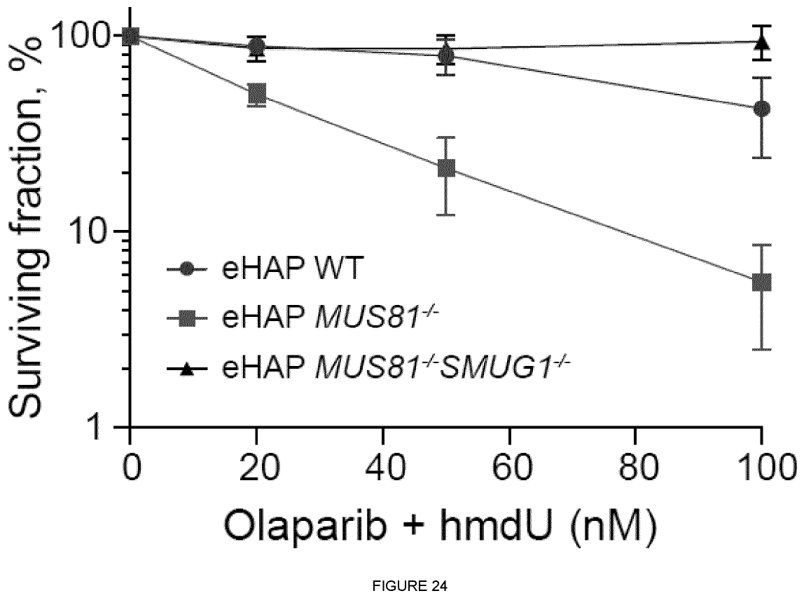

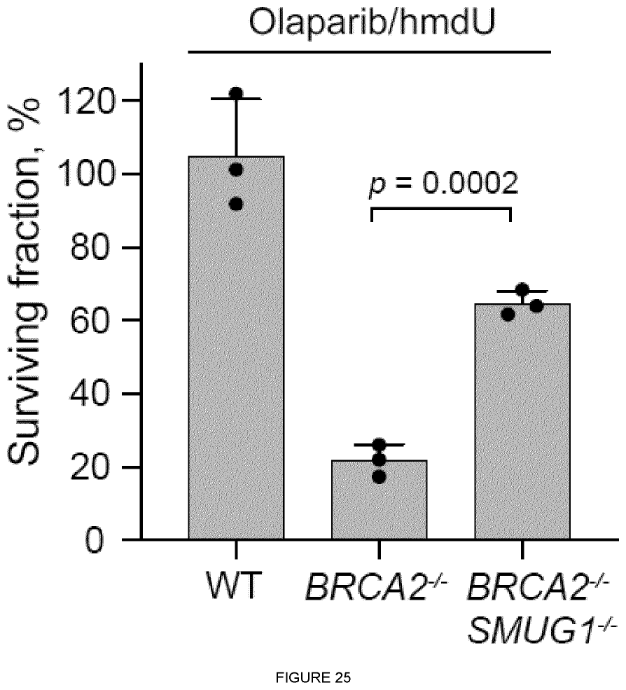

From the initial screen we identified another BER factor that when lost promoted resistance to olaparib treatment or hmdC treatment in HR-defective cells (FIG. 1), Single-Strand-Selective Monofunctional Uracil-DNA Glycosylase 1 (SMUG1). SMUG1 is a DNA glycosylase involved in BER repair by recognising and excising aberrant nucleotides from genomic DNA (Olinski et al 2016, PMID: 27036066). Interestingly, the main target of SMUG1 is hmdU, indicating that the observed resistance to olaparib could be due to endogenously present hmdU. To further explore this possibility, we generated SMUG1 k/o cells and found that whereas MUS81 k/o's were hypersensitive to hmdU/olaparib treatment, MUS81/SMUG1 k/o's were completely resistant (FIG. 24). Complementation of the double k/o with wild-type SMUG1, but not catalytically inactive SMUG1G87Y, restored cell death upon hmdU/olaparib treatment. Taken together, these results show that SMUG1-dependent excision of genomic hmdU is the underlying basis for synthetic lethality. Similar observations were made in DLD1 BRCA2 deficient cells following SMUG1 disruption (FIG. 25).

hmdU/Olaparib Treatment Promotes PARP Trapping, DNA Damage Signalling and Apoptosis in a SMUG1 Dependent Manner

PARP inhibitor induced cytotoxicity is thought to be at least partly due to trapping of PARP at sites of SSB, ribonucleotides and other BER repair intermediates. To address if olaparib is trapping PARP at sites of hmdU in genomic DNA, we carried out cellular fractionation and analysed the chromatin fraction for PARP1.

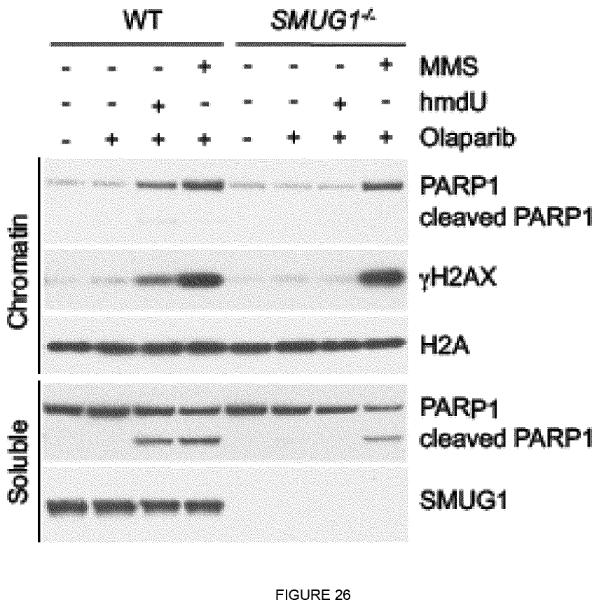

Following hmdU/olaparib treatment, we observed an increased chromatin association of PARP1 compared to treatment with olaparib alone, in conjunction with induction of DNA breakage, as evidenced by phosphorylation of gH2AX, and apoptosis as evidenced by PARP cleavage (FIG. 12C). Strikingly, the olaparib induced PARP trapping, DNA damage and apoptosis was completely rescued by ablation of SMUG1 expression. In contrast, we did not observe any difference in PARP trapping or DNA damage signaling between WT and SMUG1-deficient cells treated with the alkylating agent methyl methanesulfonate (MMS) (FIG. 26), confirming that the lack of PARP trapping in SMUG1-deficient cells was not due to a general BER defect.

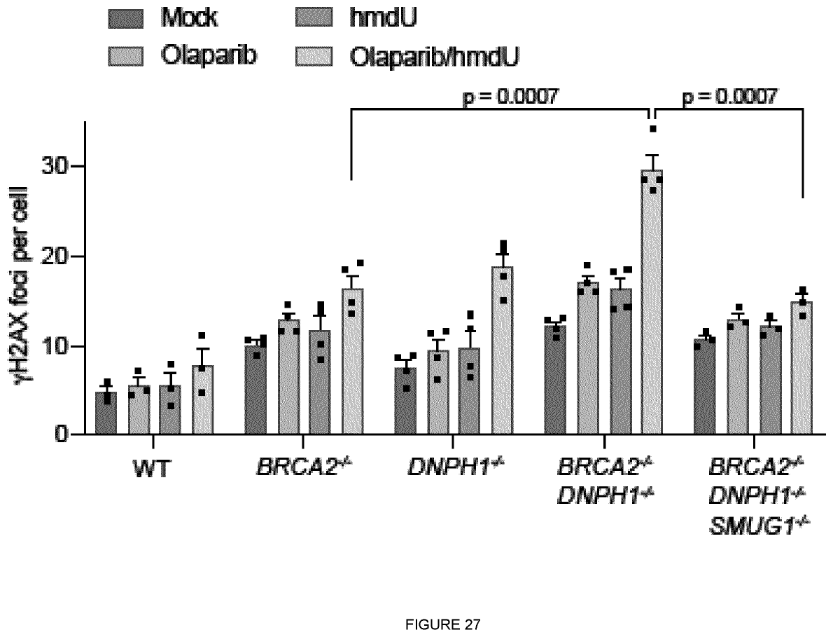

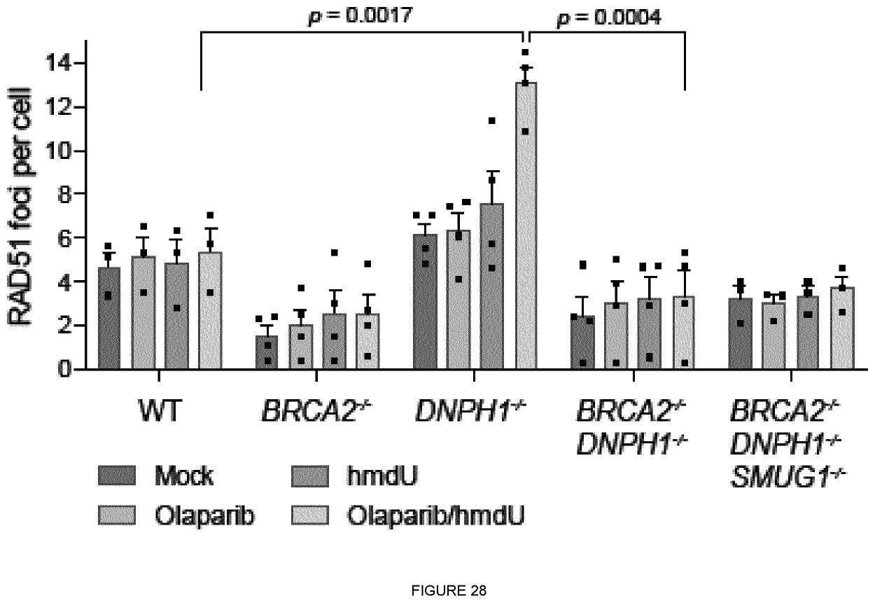

We next analyzed DSB formation following hmdU/olaparib co-treatment and observed low but reproducible levels of DNA breakage in DLD1 BRCA2-deficient cells as compared to wild-type. This was accompanied by phosphorylation of gH2AX, as shown by immunoblotting and immunofluorescence (FIG. 27). We also observed CHK1 phosphorylation and DSB formation, which are characteristic of replication stress and fork breakage (L. Toledo, et al Mol. Cell 66, 735-749 (2017)). However, upon disruption of DNPH1, we found a dramatic increase in DSB formation, checkpoint signaling (KAP1 phosphorylation), replication stress (CHK1 phosphorylation) and apoptosis (PARP1 cleavage), phenotypes that were completely rescued by disruption of SMUG1. Our data demonstrate that SMUG1-induced replication fork breakage and DSB formation is an underlying cause of PARPi-induced synthetic lethality brought about by PARP trapping at sites of hmdU excision. As expected, PARP trapping, induced by hmdU/olaparib treatment, led to a significant increase in the number of RAD51 foci (FIG. 28).

Taken together, our data suggests that SMUG1 detects and excises genomic incorporated hmdU which initiates the BER pathway. Upon PARP inhibition, PARP becomes trapped at the DNA repair intermediates during the excision of hmdU thereby promoting to a cytotoxic response, presumable through replication fork collapse. The fact that HR is essential for the restart of collapsed replication forks, likely explains the synthetic lethality observed in HR deficient cells.

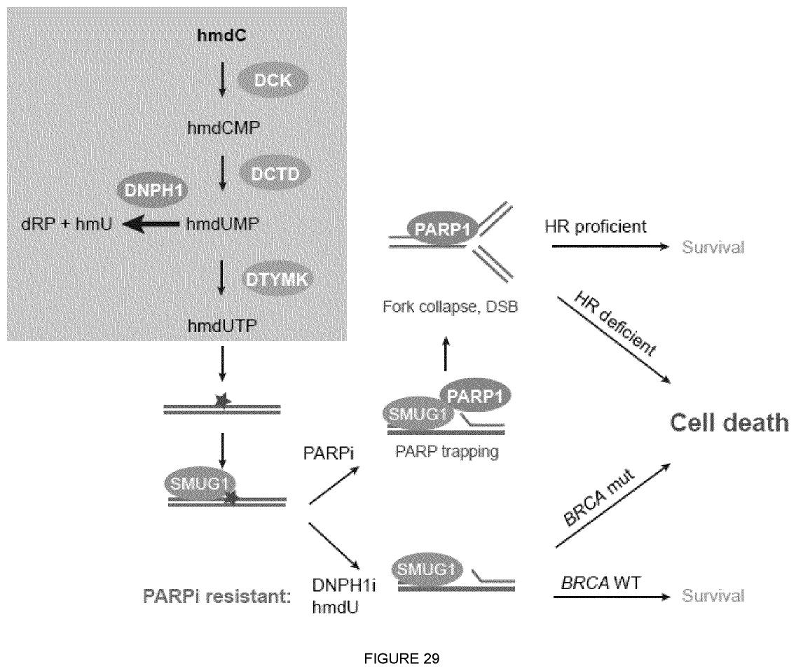

Rapidly proliferating cancer cells are dependent on a steady supply of nucleotides which is achieved by de novo synthesis and salvage pathways that are upregulated in many cancers. The re-incorporation of nucleotides carrying epigenetic marks, such as hmdCMP, is undesirable due to their potential to alter gene expression. Our results defined a novel metabolic pathway whereby cells eliminate epigenetically modified hmdCMP in a two-step process entailing deamination to cytotoxic hmdUMP by DCTD, followed by DNPH1 dependent hydrolysis into hmU and dRP (FIG. 29). As DNPH1 is a c-Myc targeted gene, its expression is directly linked to activation of the nucleotide salvage pathway (J. T. Cunningham, et al Cell 157, 1088-1103 (2014)) and as such could help cancer cells cope with increased hmdUMP levels.

Although our results indicate that hmdU(MP) arises from DCTD and not CDA dependent deamination as previously reported (Zauri et al Nature 524, 114-118 (2015)), it is possible that the increased CDA-dependent production of hmdU and hmdC observed in several breast and pancreatic cancer cells might result from a gain-of-function activity. Consistent with this, high DCTD expression correlates with poor prognosis in malignant glioma patients (Hu et al Sci. Rep. 7, 11568 (2017)). We therefore speculate that cancers with high expression of CDA or DCTD may depend on DNPH1 for survival due to increased levels of cytotoxic hmdUMP. As such, they could be used as biomarkers for DNPH1 i therapy.

In addition to PARP trapping at BER intermediates (J. Murai et al. Cancer Res. 72, 5588-5599 (2012); M. Zimmermann et al Nature 559, 285-289 (2018)), several new hypotheses have been proposed to account for the underlying mechanism of PARPi killing of HR deficient cells, such as increased fork speed (A. Maya-Mendoza et al., Nature 559, 279-284 (2018)), persistent single-strand breaks at unligated Okazaki fragments (H. Hanzlikova et al., Mol. Cell 71, 319-333 (2018)) and/or the formation replication-associated ssDNA gaps (33). Although the present work does not allow us to distinguish between these possibilities, we found that loss of either PARP1 or PARG suppressed PARPi synthetic lethality, in agreement with previous reports (Murai et al supra; Pettit et al Nat. Commun. 9, 1849 (2018)). Importantly, however, hdU/PARPi treatment led to SMUG1-dependent PARP trapping followed by replication fork collapse, DSB formation and apoptosis, consistent with PARPi cytotoxicity being mediated both by PARP trapping and non-trapping events.

In summary, we have shown that hmdU is an endogenous DNA lesion that potentiates the response to PARPi therapy. Furthermore, we discovered that PARPi-resistant BRCA1-defective cells with loss of either PARP1 or 53BP1, were effectively killed by hmdU/DNPH1i treatment. As 53BP1 loss restores HR-proficiency through reactivation of DNA end resection (J. E. Jaspers et al. Cancer Discov. 3, 68-81 (2013); S. F. Bunting et al. Cell 141, 243-254 (2010)), these data indicate that BRCA1's role in mediating fork protection is a key event in safeguarding against hmdU/DNPH1i-induced cell death (M. Daza-Martin et al. Nature 571, 521-527 (2019); K. P. Bhat et al. Nat. Struct. Mol. Biol. 25, 446-453 (2018)). The role of DNPH1 and hmdU in the sensitization of HR-deficient cancer cells to PARPi indicates that DNPH1 should be investigated as a high-priority potential druggable target.

```
Sequences
SEQ ID NO: 1
  001 MAAAMVPGRS ESWERGEPGR PALYFCGSIR GGREDRTLYE RIVSRLRRFG TVLTEHVAAA
  061 ELGARGEEAA GGDRLIHEQD LEWLQQADVV VAEVTQPSLG VGYELGRAVA FNKRILCLFR
  121 PQSGRVLSAM IRGAADGSRF QVWDYEEGEV EALLDRYFEA DPPGQVAASP DPTT
(DNPH1 homo sapiens; NP_006434.1)
SEQ ID NO: 2
  001 GCCGGAGAGC GCGGGCGGCT GGGGAATGGC TGCTGCCATG GTGCCGGGGC GCAGCGAGAG
  061 CTGGGAGCGC GGGGAGCCTG GCCGCCCGGC CCTGTACTTC TGCGGGAGCA TTCGCGGCGG
  121 AGGCGAGGAC AGGACGCTGT ACGAGCGGAT CGTGTCTCGG CTGCGGCGAT TCGGGACAGT
  181 GCTCACCGAG CACGTGGCGG CCGCCGAGCT GGGCGCGCGC GGGGAAGAGG CTGCTGGGGG
  241 TGACAGGCTC ATCCATGAGC AGGACCTGGA GTGGCTGCAG CAGGCGGACG TGGTCGTGGC
  301 AGAAGTGACA CAGCCATCCT TGGGTGTAGG CTATGAGCTG GGCCGGGCCG TGGCCTTTAA
  361 CAAGCGGATC CTGTGCCTGT TCCGCCCGCA GTCTGGCCGC GTGCTTTCGG CCATGATCCG
  421 GGGAGCAGCA GATGGCTCTC GGTTCCAGGT GTGGGACTAT GAGGAGGGAG AGGTGGAGGC
  481 CCTGCTGGAT CGATACTTCG AGGCTGATCC TCCAGGGCAG GTGGCTGCCT CCCCTGACCC
  541 AACCACTTGA CTTAATCTCA CTTTCTTAAA TTCTTCTATT CTCAGACACT GCTCTAGTAC
  601 CATTCCTTCC TCTTAGCCCC AGGAGCAAAT TAAAAGGTAC AGTTAAAATC CTAA
(DNPH1 homo sapiens; NM_006443.3)
SEQ ID NO: 3
  001 MPQAFLLGSI HEPAGALMEP QPCPGSLAES FLEEELRLNA ELSQLQFSEP VGIIYNPVEY
  061 AWEPHRNYVT RYCQGPKEVL FLGMNPGPFG MAQTGVPFGE VSMVRDWLGI VGPVLTPPQE
  121 HPKRPVLGLE CPQSEVSGAR FWGFFRNLCG QPEVFFHHCF VHNLCPLLFL APSGRNLTPA
  181 ELPAKQREQL LGICDAALCR QVQLLGVRLV VGVGRLAEQR ARRALAGLMP EVQVEGLLHP
  241 SPRNPQANKG WEAVAKERLN ELGLLPLLLK
(SMUG1 homo sapiens; NP_001230716.1)
SEQ ID NO: 4
      1 GGGTGGGGGA AAGGAACCGG AAACGGGATG GGGAGCTGGA CCAGATTATG AGGTTACAGA
    61 AAGCCTGGCC TACATTTTAC TCTTTTTGGA TTTCTTCCTC ATCAAGAGAC TGCTGCAGTG
  121 CCTGTCATGT GACAGCGGCA TGGACATATG CCCCAGGCTT TCCTGCTGGG GTCCATCCAT
  181 GAGCCTGCAG GTGCCCTCAT GGAGCCCCAG CCCTGCCCTG GAAGCTTGGC TGAGAGCTTC
  241 CTGGAGGAGG AGCTTCGGCT CAATGCTGAG CTGAGCCAGC TGCAGTTTTC GGAGCCTGTG
  301 GGCATCATCT ACAATCCCGT GGAGTATGCA TGGGAGCCAC ATCGCAACTA CGTGACTCGC
  361 TACTGCCAGG GCCCCAAGGA AGTACTCTTC CTGGGCATGA ACCCTGGACC TTTTGGCATG
  421 GCCCAGACTG GGGTGCCCTT TGGGGAAGTA AGCATGGTCC GGGACTGGTT GGGCATTGTG
  481 GGGCCTGTGC TGACCCCTCC CCAAGAGCAT CCTAAAGGAC CAGTGCTGGG ACTGGAGTGC
  541 CCACAGTCAG AAGTGAGTGG TGCCCGATTC TGGGGCTTTT TCCGGAACCT CTGTGGACAG
  601 CCTGAGGTCT TCTTCCATCA CTGTTTTGTC CACAATCTAT GCCCTCTGCT TTTCCTGGCT
  661 CCCAGCGGGC GCAACCTTAC TCCTGCTGAG CTGCCTGCCA AGCAGCGAGA ACAGCTTCTT
  721 GGGATCTGTG ATGCAGCCCT CTGCCGGCAG GTGCAGCTGC TGGGGGTGCG GCTGGTGGTG
  781 GGAGTTGGGC GACTGGCAGA GCAGCGGGCA CGACGGGCTC TGGCAGGCCT GATGCCAGAG
  841 GTCCAGGTGG AAGGGCTCCT GCATCCCTCT CCCCGTAACC CACAGGCCAA CAAGGGCTGG
  901 GAGGCAGTGG CCAAGGAAAG ATTGAATGAG CTGGGGCTGC TGCCACTGCT GTTGAAATGA
  961 GTGCCCTTGG GGCCTTGCAT GGGACACATT CAAGACCTCG AAGTCATTCT TGGCCAAGCA
1021 GATGACAACA CATCTCCTGG ACTGGAGCAA AAGGTCCTTC TGTGCACCCT GGTCGCTGGG
1081 AAACGTATTC TTTGATCTGT TGAACTGTCT TCCAACCTGC CATGGCAGTT TTGACACTAC
1141 TCCTGTTTGC CCTCCTGATT CCTGCTTTCT TTACCTTTTA ACATTGCCCC TTTCAGGGGA
1201 CCCCACTTTG TAGGGAATCT GCAGAAGGTG TGCTTTTGCA CTTGCAGACT GCTCTACCTC
1261 AGTGTTTCCT TGGGAGACTT TATTCAGCTG AGAGTGCCCT AGACAGTAAC TTCTAAGGTC
1321 ACGTTTACTA TTTCAGAGGA AATATCTTGC CAGGATACCT ACCCATCCTT ATAGAACAGT
1381 TACCTTTAGC TGACCCCTTT CCTCACAGGG ACCAAGACAA AGCATGGGAC ATGAAATTAA
1441 GAGTGAACTT CTTATGGGAG GCTGCAGCTG GATCAGAGGA AAAATCCAGT GTGACAGAGT
1501 GCAAGTCAGA AGACCTGGCT TTTCATCCCA GCTTTGAAAC TTGGAACTTT TTGATTGACA
1561 AATTAATAAA CCTCTCTATG CCTCAGGCTC CTCATCTGTA AAACAGGGAT GCCTACCTTA
1621 TGGGGTTGGT GTGAGGATTA ACTGAGATCA CACTTGAAAG TGCCTTGCAT GGGCTTGGCA
1681 TGTGGGTGCT CATTACATAG TCTTCTTGTT TTCTTGCCTC TGGGTTGAGG ATTCACAGCA
1741 CTGCACTACA AGGGTAGCCC CTCTCCATTC TGGATACCTT GCTAGGAGAG AGTCTGGGCT
1801 TCCACCTGCG GCCAGGGTGA AGGGAAAATT CTTGCCATGT CAGAGATGGA TCAATGACTA
1861 TGAAAAAACT AGCCTGTGAT TTTTTTTCAC TCTAAGCATT AAGAGCTCCT AAAGCCTTTT
1921 GTCTTGTTAC TTTTTTCCCC CTCTTGCATG CTAGAGGTGG AAGGTAGTCA TTTAGATTTC
1981 CCCCCTCTTC CCTCTTCATT GTTGGCCAGG GGTTCTCTTG GGCAGCTGGG GGAAGGATGT
2041 AGTAGATCTC TCTTTTTTTT TTTTTTTTGA GATGGAGTCT CATGCTGTTG CCCAGGCTGG
2101 AGTGCAGTGG CATGGTCTCA GTTCACTGCA ACATCTGCTT TCCCGGTTCA AGTGATTCTC
2161 CTGCCTCAGC CTCCTGTATA GCTGGAATTA CAGGCATGTG CCACCACGCC TGGCTAATTT
2221 TTGTATTTTT ATTAAAGACA GTGTTTCACC ATGTTGGCCA GGCTGGTCTC AAACTCCTGA
2281 CTTCAAGTAA TCCACCTGTC TTGGCCTCCC AAAGTGCTGC TGCCTGGCTG GATGTAGCAG
2341 ATCTCCAATT GCCTTACCAA ACCTGCAGGG TCTCTGTTAA GTGTCTGTTA GGGGTCTTAT
2401 ATGATACTCT GGACACCGTG AGAAACAGGT GATGGGGCAG ATGGGAACCT GGTATCCAAC
2461 TCATTTTTTA TGTCTGCTGA ATGGTTGACT GGGTGCCCCT TTGGTAAGGG CTGGGAAAGT
2521 GAACCAAGTT GGGAGCAGAG CAGGTCTTGA GACCACCTTA ATGAGGAACA CATGGTCTAC
2581 TTCCTATCTC CTGGGACACA GGTCATTTCC AGCTTCCAGA ATTAGTAAGA TTTTTCATAT
2641 AAATCCTCCC CTACTTCATT GTATAATAAA GAATTTGGCC TTTGTCTTCA GTTCCTGAAA
2701 AAAAAAAAAA AAAAAA
(SMUG1 homo sapiens; NM_001243787.1)
```

1. A method of treating an HR deficient cancer comprising;
administering a PARP inhibitor to an individual in need thereof and reducing 2′-deoxynucleoside 5′-phosphate N-hydrolase 1 (DNPH1) activity in the individual

2. A method of sensitising a HR deficient cancer in an individual to treatment with a PARP inhibitor comprising;
reducing DNPH1 activity in the individual,
wherein said reducing sensitises the HR deficient cancer to treatment with the PARP inhibitor.

3. A method of sensitising a HR deficient cancer in an individual to reduced or deficient 2′-deoxynucleoside 5′-phosphate N-hydrolase 1 (DNPH1) expression or activity comprising;
administering a PARP inhibitor to the individual,
wherein said administering sensitises the HR deficient cancer to reduced or deficient 2′-deoxynucleoside 5′-phosphate N-hydrolase 1 (DNPH1) expression or activity.

4. A method of treating an HR deficient cancer comprising;
administering a combination of PARP inhibitor and a 5-modified-2′-deoxypyrimidine nucleoside or to an individual in need thereof.

5. A method of sensitising a HR deficient cancer in an individual to treatment with a PARP inhibitor comprising:
administering a 5-modified-2′-deoxypyrimidine nucleoside to the individual,
wherein said administering sensitises the HR deficient cancer to treatment with the PARP inhibitor

6. A method of sensitising a HR deficient cancer in an individual to treatment with a 5-modified-2′-deoxypyrimidine nucleoside comprising;
administering a PARP inhibitor to the individual,
wherein said administering sensitises the HR deficient cancer to treatment with a 5-modified-2′-deoxypyrimidine nucleoside.

7. A method of treating an HR deficient cancer comprising;
administering a 5-modified-2′-deoxypyrimidine nucleoside to an individual in need thereof and reducing DNPH1 activity in the individual

8. A method of sensitising a HR deficient cancer in an individual to treatment with a 5-modified-2′-deoxypyrimidine nucleoside comprising;
reducing DNPH1 activity in the individual,
wherein said reducing sensitises the HR deficient cancer to treatment with the a 5-modified-2′-deoxypyrimidine nucleoside.

9. A method of sensitising a HR deficient cancer in an individual to reduced 2′-deoxynucleoside 5′-phosphate N-hydrolase 1 (DNPH1) expression or activity comprising;
administering a 5-modified-2′-deoxypyrimidine nucleoside to the individual,
wherein said administering sensitises the HR deficient cancer to reduced or deficient 2′-deoxynucleoside 5′-phosphate N-hydrolase 1 (DNPH1) expression or activity.

10. A method according to any one of claims 7 to 9 wherein the HR deficient cancer is resistant to treatment with a PARP inhibitor.

11. A method of treating an HR deficient cancer comprising;
administering a PARP inhibitor and a 5-modified-2′-deoxypyrimidine nucleoside to an individual in need thereof and reducing DNPH1 activity in the individual

12. A method of sensitising a HR deficient cancer in an individual to treatment with a PARP inhibitor comprising;
administering a 5-modified-2′-deoxypyrimidine nucleoside to the individual and reducing DNPH1 activity in the individual,
wherein said administering and reducing sensitises the HR deficient cancer to treatment with the PARP inhibitor.

13. A method of ameliorating toxicity in an organ or tissue of an individual undergoing treatment with a PARP inhibitor comprising;
selectively reducing SMUG1 activity in the organ or tissue of the individual.

14. A method according to claim 13 wherein the individual has an HR deficient cancer.

15. A method according to any one of the preceding claims wherein the PARP inhibitor is selected from the group consisting of olaparib, rucaparib, niraparib, talazoparib, velaparib, pamiparib (BGB-290), CEP-9722 (11-methoxy-2-((4-methylpiperazin-1-yl)methyl)-4,5,6,7-tetrahydro-1H-cyclopenta[a]pyrrolo[3,4-c]carbazole-1,3(2H)-dione) and E7016 (10-((4-Hydroxypiperidin-1-yl)methyl)chromeno[4,3,2-de]phthalazin-3(2H)-one).

16. A method according to any one of the preceding claims wherein the HR deficient cancer is deficient in an HR gene selected from BRCA1, BRCA2, MUS81, RAD52, RAD51, RAD50, ATM/ATR, FANC, BARD1, BRIP1, CHEK1, CHEK2, FAM175A, NBN, PALB2, MRE11A, NBS1, RBBP8 (CtIP), MRE11, RPA, MMR, H2AX and TP53.

17. A method according to any one of the preceding claims wherein DNPH1 activity is reduced by administering to the individual an agent which reduces DNPH1 activity.

18. A method according to claim 17 wherein the agent is a DNPH1 inhibitor.

19. A method according to claim 18 wherein the DNPH1 inhibitor is an organic compound having a molecular weight of 900 Da or less.

20. A method according to claim 19 wherein the DNPH1 inhibitor is a nucleoside or nucleotide analogue.

21. A method according to claim 20 wherein the DNPH1 inhibitor is N6BA or a pharmaceutically acceptable salt, solvate, or analogue thereof.

22. A method according to claim 17 wherein the agent is a suppressor nucleic acid that reduces expression of active DNPH1 polypeptide.

23. A method according to claim 22 wherein the suppressor nucleic acid is a siRNA or shRNA.

24. A method according to claim 23 wherein the suppressor nucleic acid comprises a nucleotide sequence at least 95% identical to a contiguous sequence of 15 to 40 nucleotides of SEQ ID NO: 2.

25. A method according to claim 22 wherein the suppressor nucleic acid is an antisense oligonucleotide.

26. A method according to claim 17 wherein the agent is a targeted nuclease that reduces expression of active DNPH1 polypeptide to the individual.

27. A method according to claim 26 wherein the targeted nuclease is a ZFN, TALEN or meganuclease that recognises a target sequence within the DNPH1 gene.

28. A method according to claim 26 wherein the targeted nuclease is a CRISPR associated nuclease, said CRISPR associated nuclease being administered in combination with a guide RNA that recognises a target sequence within the DNPH1 gene.

29. A method according to any one of claims 26-28 wherein the targeted nuclease cleaves genomic DNA at the target sequence of the DNPH1 gene, thereby causing a deletion or insertion which reduces expression of active DNPH1 polypeptide.

30. A method of screening for a compound useful in sensitising an HR deficient cancer in a patient to treatment with a PARP inhibitor or a 5-modified-2′-deoxypyrimidine nucleoside, the method comprising;
determining the activity of DNPH1 in the presence or absence of a test compound,
wherein a decrease in DNPH1 activity in the presence relative to the absence of the test compound is indicative that the test compound is a candidate compound for use in sensitising an HR deficient cancer in a patient to treatment with a PARP inhibitor.

31. A method of screening for a compound useful in sensitising an HR deficient cancer in a patient to treatment with a PARP inhibitor or a 5-modified-2′-deoxypyrimidine nucleoside, the method comprising;
determining the expression of DNPH1 in a mammalian cell in the presence or absence of a test compound,
wherein a decrease in DNPH1 expression in the presence relative to the absence of the test compound is indicative that the test compound is a candidate compound for use in sensitising an HR deficient cancer in a patient to treatment with a PARP inhibitor.

32. A method of screening for a compound useful in ameliorating toxicity in individual undergoing treatment with a PARP inhibitor comprising
determining the activity of SMUG1 in the presence or absence of a test compound,
wherein a decrease in SMUG1 activity in the presence relative to the absence of the test compound is indicative that the test compound is useful in ameliorating toxicity in individual undergoing treatment with a PARP inhibitor.

33. A method of screening for a compound useful in ameliorating toxicity in individual undergoing treatment with a combination of a PARP inhibitor and a 5-modified-2′-deoxypyrimidine nucleoside comprising
determining the expression of SMUG1 in the presence or absence of a test compound,
wherein a decrease in SMUG1 expression in the presence relative to the absence of the test compound is indicative that the test compound is useful in ameliorating toxicity in individual undergoing treatment with a combination of a PARP inhibitor and a 5-modified-2′-deoxypyrimidine nucleoside.

34. A method according to claim 33 wherein the method further comprises determining the effect of the test compound on the expression or activity of SMUG1 in target organ or tissue relative to a non-target organ or tissue.

35. A method of predicting the response of an HR deficient cancer in an individual to a combination of active agents, selected from (i) a PARP inhibitor and an agent that reduces DNPH1 activity; (ii) a PARP inhibitor and a 5-modified-2′-deoxypyrimidine nucleoside, (iii) an agent that reduces DNPH1 activity and a 5-modified-2′-deoxypyrimidine nucleoside and (iv) a PARP inhibitor, an agent that reduces DNPH1 activity and a 5-modified-2′-deoxypyrimidine nucleoside, wherein the method comprises;
determining the expression of SMUG1 in a sample of HR deficient cancer cells obtained from the individual,
wherein a decrease in SMUG1 expression in the cancer cells relative to control cells may be indicative that the HR deficient cancer is insensitive or has reduced sensitivity to the combination of active agents and/or the individual is not responsive to the combination.

36. A method of predicting the response of an HR deficient cancer in an individual to a combination of active agents, selected from (i) a PARP inhibitor and an agent that reduces DNPH1 activity; (ii) a PARP inhibitor and a 5-modified-2′-deoxypyrimidine nucleoside, (iii) an agent that reduces DNPH1 activity and a 5-modified-2′-deoxypyrimidine nucleoside and (iv) a PARP inhibitor, an agent that reduces DNPH1 activity and a 5-modified-2′-deoxypyrimidine nucleoside, wherein the method comprises;
determining the expression of DNPH1 in a sample of HR deficient cancer cells obtained from the individual,
wherein a decrease in DNPH1 expression in the cancer cells relative to control cells may be indicative that the HR deficient cancer is sensitive or has increased sensitivity to the combination of active agents relative to control cells and/or the individual is responsive to the combination.

37. A method of predicting the response of an HR deficient cancer in an individual to a combination of active agents, selected from (i) a PARP inhibitor and an agent that reduces DNPH1 activity; (ii) a PARP inhibitor and a 5-modified-2′-deoxypyrimidine nucleoside, (iii) an agent that reduces DNPH1 activity and a 5-modified-2′-deoxypyrimidine nucleoside and (iv) a PARP inhibitor, an agent that reduces DNPH1 activity and a 5-modified-2′-deoxypyrimidine nucleoside, wherein the method comprises;
determining the intracellular concentration of hmdU in a sample of HR deficient cancer cells obtained from the individual,
wherein an increase in intracellular concentration of hmdU in the cancer cells relative to control cells may be indicative that the HR deficient cancer is sensitive or has increased sensitivity to the combination of active agents relative to control cells and/or the individual is responsive to the combination.

38. A method of selecting an individual with an HR deficient cancer as likely to respond to therapy with a combination of active agents, selected from (i) a PARP inhibitor and an agent that reduces DNPH1 activity; (ii) a PARP inhibitor and a 5-modified-2′-deoxypyrimidine nucleoside, (iii) an agent that reduces DNPH1 activity and a 5-modified-2′-deoxypyrimidine nucleoside and (iv) a PARP inhibitor, an agent that reduces DNPH1 activity and a 5-modified-2′-deoxpyrimidine nucleoside, wherein the method comprises;
determining the expression of SMUG1 in a sample of HR deficient cancer cells obtained from the individual, and
selecting the individual as likely to respond to therapy where the expression of SMUG1 is not decreased relative to control cells.

39. A method of selecting an individual with an HR deficient cancer as likely to respond to therapy with a combination of active agents, selected from (i) a PARP inhibitor and an agent that reduces DNPH1 activity; (ii) a PARP inhibitor and a 5-modified-2′-deoxypyrimidine nucleoside, (iii) an agent that reduces DNPH1 activity and a 5-modified-2′-deoxypyrimidine nucleoside and (iv) a PARP inhibitor, an agent that reduces DNPH1 activity and a 5-modified-2′-deoxpyrimidine nucleoside, wherein the method comprises;
determining the expression of DNPH1 in a sample of HR deficient cancer cells obtained from the individual, and
selecting the individual as likely to respond to therapy where the expression of DNPH1 in the cancer cells is decreased relative to control cells.

40. A method of selecting an individual with an HR deficient cancer as likely to respond to therapy with a combination of active agents, selected from (i) a PARP inhibitor and an agent that reduces DNPH1 activity; (ii) a PARP inhibitor and a 5-modified-2′-deoxypyrimidine nucleoside, (iii) an agent that reduces DNPH1 activity and a 5-modified-2′-deoxypyrimidine nucleoside and (iv) a PARP inhibitor, an agent that reduces DNPH1 activity and a 5-modified-2′-deoxypyrimidine nucleoside, wherein the method comprises;
determining the intracellular concentration of hmdU in a sample of HR deficient cancer cells obtained from the individual, and
selecting the individual as likely to respond to therapy where the intracellular concentration of hmdU in the cancer cells is increased relative to control cells.

41. A method according to any of claims 38 to 40, wherein the method further comprises:
administering the combination of active agents to the individual selected as likely to respond to therapy.


SEQUENCE LISTING (7 sequences)

SEQ ID NO: 1 - protein, 174 aa, Homo sapiens
MAAAMVPGRSESWERGEPGRPALYFCGSIRGGREDRTLYERIVSRLRRFGTVLTEHVAAAELGARGEEAAGGDRLIHEQDLEWLQQADVVVAEVTQPSLGVGYELGRAVAFNKRILCLFRPQSGRVLSAMIRGAADGSRFQVWDYEEGEVEALLDRYFEADPPGQVAASPDPTT

SEQ ID NO: 2 - DNA, 654 bp, Homo sapiens
gccggagagcgcgggcggctggggaatggctgctgccatggtgccggggcgcagcgagagctgggagcgcggggagcctggccgcccggccctgtacttctgcgggagcattcgcggcggacgcgaggacaggacgctgtacgagcggatcgtgtctcggctgcggcgattcgggacagtgctcaccgagcacgtggcggccgccgagctgggcgcgcgcggggaagaggctgctgggggtgacaggctcatccatgagcaggacctggagtggctgcagcaggcggacgtggtcgtggcagaagtgacacagccatccttgggtgtaggctatgagctgggccgggccgtggcctttaacaagcggatcctgtgcctgttccgcccgcagtctggccgcgtgctttcggccatgatccggggagcagcagatggctctcggttccaggtgtgggactatgaggagggagaggtggaggccctgctggatcgatacttcgaggctgatcctccagggcaggtggctgcctcccctgacccaaccacttgacttaatctcactttcttaaattcttctattctcagacactgctctagtaccattccttcctcttagccccaggagcaaattaaaaggtacagttaaaatcctaa

SEQ ID NO: 3 - protein, 270 aa, Homo sapiens
MPQAFLLGSIHEPAGALMEPQPCPGSLAESFLEEELRLNAELSQLQFSEPVGIIYNPVEYAWEPHRNYVTRYCQGPKEVLFLGMNPGPFGMAQTGVPFGEVSMVRDWLGIVGPVLTPPQEHPKRPVLGLECPQSEVSGARFWGFFRNLCGQPEVFFHHCFVHNLCPLLFLAPSGRNLTPAELPAKQREQLLGICDAALCRQVQLLGVRLVVGVGRLAEQRARRALAGLMPEVQVEGLLHPSPRNPQANKGWEAVAKERLNELGLLPLLLK

SEQ ID NO: 4 - DNA, 2716 bp, Homo sapiens
gggtgggggaaaggaaccggaaacgggatggggagctggaccagattatgagcttacagaaagcctggcctacattttactctttttggatttcttcctcatcaagagactgctgcagtgcctgtcatgtgacagcggcatggacatatgccccaggctttcctgctggggtccatccatgagcctgcaggtgccctcatggagccccagccctgccctggaagcttggctgagagcttcctggaggaggagcttcggctcaatgctgagctgagccagctgcagttttcggagcctgtgggcatcatctacaatcccgtggagtatgcatgggagccacatcgcaactacgtgactcgctactgccagggccccaaggaagtactcttcctgggcatgaaccctggaccttttggcatggcccagactggggtgccctttggggaagtaagcatggtccgggactggttgggcattgtggggcctgtgctgacccctccccaagagcatcctaaacgaccagtgctgggactggagtgcccacagtcagaagtgagtggtgcccgattctggggctttttccggaacctctgtggacagcctgaggtcttcttccatcactgttttgtccacaatctatgccctctgcttttcctggctcccagcgggcgcaaccttactcctgctgagctgcctgccaagcagcgagaacagcttcttgggatctgtgatgcagccctctgccggcaggtgcagctgctgggggtgcggctggtggtgggagttgggcgactggcagagcagcgggcacgacgggctctggcaggcctgatgccagaggtccaggtggaagggctcctgcatccctctccccgtaacccacaggccaacaagggctgggaggcagtggccaaggaaagattgaatgagctggggctgctgccactgctgttgaaatgagtgcccttggggccttgcatgggacacattcaagacctcgaagtcattcttggccaagcagatgacaacacatctcctggactggagcaaaaggtccttctgtgcaccctggtcgctgggaaacgtattctttgatctgttgaactgtcttccaacctgccatggcagttttgacactactcctgtttgccctcctgattcctgctttctttaccttttaacattgcccctttcaggggaccccactttgtagggaatctgcagaaggtgtgcttttgcacttgcagactgctctacctcagtgtttccttgggagactttattcagctgagagtgccctagacagtaacttctaaggtcacgtttactatttcagaggaaatatcttgccaggatacctacccatccttatagaacagttacctttagctgacccctttcctcacagggaccaagacaaagcatgggacatgaaattaagagtgaacttcttatgggaggctgcagctggatcagaggaaaaatccagtgtgacagagtgcaagtcagaagacctggcttttcatcccagctttgaaacttggaactttttgattgacaaattaataaacctctctatgcctcaggctcctcatctgtaaaacagggatgcctaccttatggggttggtgtgaggattaactgagatcacacttgaaagtgccttgcatgggcttggcatgtgggtgctcattacatagtcttcttgttttcttgcctctgggttgaggattcacagcactgcactacaagggtagcccctctccattctggataccttgctaggagagagtctgggcttccacctgcggccagggtgaagggaaaattcttgccatgtcagagatggatcaatgactatgaaaaaactagcctgtgattttttttcactctaagcattaagagctcctaaagccttttgtcttgttacttttttccccctcttgcatgctagaggtggaaggtagtcatttagatttcccccctcttccctcttcattgttggccaggggttctcttgggcagctgggggaaggatgtagtagatctctcttttttttttttttttgagatggagtctcatgctgttgcccaggctggagtgcagtggcatggtctcagttcactgcaacatctgctttcccggttcaagtgattctcctgcctcagcctcctgtatagctggaattacaggcatgtgccaccacgcctggctaatttttgtatttttattaaagacagtgtttcaccatgttggccaggctggtctcaaactcctgacttcaagtaatccacctgtcttggcctcccaaagtgctgctgcctggctggatgtagcagatctccaattgccttaccaaacctgcagggtctctgttaagtgtctgttaggggtcttatatgatactctggacaccgtgagaaacaggtgatggggcagatgggaacctggtatccaactcattttttatgtctgctgaatggttgactgggtgcccctttggtaagggctgggaaagtgaaccaagttgggagcagagcaggtcttgagaccaccttaatgaggaacacatggtctacttcctatctcctgggacacaggtcatttccagcttccagaattagtaagatttttcatataaatcctcccctacttcattgtataataaagaatttggcctttgtcttcagttcctgaaaaaaaaaaaaaaaaaaa

SEQ ID NO: 5 - DNA, 20 bp, Artificial Sequence
  sgRNA target sequences MUS81
tactggccagctcggcactc

SEQ ID NO: 6 - DNA, 20 bp, Artificial Sequence
  sgRNA target sequence DNPH1
tcatagcctacacccaagga

SEQ ID NO: 7 - DNA, 20 bp, Artificial Sequence
  sgRNA target sequence SMUG1
cgagtcacgtagttgcgatg